SEQUENCE LISTING (11 sequences)

SEQ ID NO: 1 - protein, 12 aa, synthetic construct
LAWSYYIGYLRL

SEQ ID NO: 2 - protein, 12 aa, synthetic construct
LASSYYSGYLRL

SEQ ID NO: 3 - protein, 25 aa, synthetic construct
YLRLILPELQARIRTYNQHYNNLLR

SEQ ID NO: 4 - protein, 27 aa, synthetic construct
IGYLRLILPELQARIRTYNQHYNNLLR

SEQ ID NO: 5 - protein, 15 aa, synthetic construct
NFNVAHGLAWSYYIG

SEQ ID NO: 6 - protein, 21 aa, synthetic construct
NFNVAHGLAWSYYIGYLRLIL

SEQ ID NO: 7 - protein, 25 aa, synthetic construct
NFNVAHGLAWSYYIGYLRLILPELQ

SEQ ID NO: 8 - protein, 32 aa, synthetic construct
NFNVAHGLAWSYYIGYLRLILPELQARIRTYN

SEQ ID NO: 9 - protein, 38 aa, synthetic construct
NFNVAHGLAWSYYIGYLRLILPELQARIRTYNQHYNNL

SEQ ID NO: 10 - protein, 39 aa, synthetic construct
NFNVAHGLAWSYYIGYLRLILPELQARIRTYNQHYNNLL

SEQ ID NO: 11 - protein, 40 aa, synthetic construct
NFNVAHGLAWSYYIGYLRLILPELQARIRTYNQHYNNLLR

(19) United States
(12) Patent Application Publication
(10) Pub. No.: US 2025/0326796 A1
(43) Pub. Date: Oct. 23, 2025

(54) PEPTIDE ANALOG WITH ANTI-INFLAMMATORY ACTIVITY AND ITS PREPARATION METHOD AND APPLICATION

(71) Applicant: Shanghai University, Shanghai (CN)

(72) Inventors: Li SU, Shanghai (CN); Honggang HU, Shanghai (CN)

(73) Assignee: Shanghai University, Shanghai (CN)

(21) Appl. No.: 18/638,826

(22) Filed: Apr. 18, 2024

(51) Int. Cl. C07K 7/08; A61K 38/00; A61P 19/02; C07K 1/06; C07K 1/20; C07K 1/34; C07K 1/36; C07K 14/00

(52) U.S. Cl. CPC C07K 7/08; A61P 19/02; C07K 1/061; C07K 1/20; C07K 1/34; C07K 1/36; C07K 14/001; A61K 38/00

(57) ABSTRACT

The present invention discloses a polypeptide compound with anti-inflammatory activity or pharmaceutically acceptable salts thereof, wherein the polypeptide compound is screened and identified from the key residue sequences of the interaction between the two in the crystal structures of the binary complex of STING. The polypeptide compound provided by the present invention can target STING to produce anti-inflammatory effects, and can be used for the treatment of gonitis and osteoarthritis.

### INCORPORATION BY REFERENCE

The sequence listing in XML format is incorporated herein by reference in its entirety.

Name of File: 7873_Sequence Listing.xml

Date of Creation: Apr. 18, 2024

Size of File: 10,798 bytes

### TECHNICAL FIELD

The present invention belongs to the field of pharmaceutical technology and specifically relates to a polypeptide compound with anti-inflammatory activity, preparation method thereof and use thereof.

### BACKGROUND ART

Osteoarthritis (OA) is a degenerative joint disease with high incidence rate and high disability rate, which has caused heavy burden to patients, families and society. It is reported that the number of OA patients worldwide has exceeded 500 million, accounting for approximately 15% of the adult population. With the intensification of population aging, the incidence of OA is gradually increasing. At present, preferred drugs for treating early-stage OA are non-steroidal anti-inflammatory drugs (NSAIDs) for local topical administration, while patients with moderate to severe OA mainly rely on oral administration of NASIDs and selective cyclooxygenase inhibitors to control symptoms with a focus on short-term benefits, as long term administration may lead to adverse effects such as cardiovascular toxicity, gastrointestinal bleeding, liver and kidney damage, etc. It is necessary to develop safe and effective drugs targeting the pathological mechanism of OA to improve the joint microenvironment, in view of the limitations of existing therapeutic drugs.

The apoptosis of articular chondrocytes and the activation of congenital inflammatory pathways play an important role in the occurrence and development of OA. STING, as a key receptor for inflammation induced aging damage in articular chondrocytes, can recognize cyclic dinucleotides in the cytoplasm, recruit TBK1 kinase and IRF3 transcription factors, phosphorylate IRF3 and form dimers, induce the expression of type I interferon and inflammatory factors, and drive inflammatory response. The absence or mutation of STING may delay the inflammatory response and cartilage degradation in spontaneous osteoarthritis, and alleviate bone and joint damage in adult mouse DMM models, indicating that STING may be a new target for treating OA. An ideal drug delivery system can improve the efficacy of drugs, and reduce dosage and toxic side effects. Polypeptide drugs, due to their wide adaptability, high safety and significant therapeutic effects, have been widely used in the treatment of diseases such as asthma, allergies and pain. There is great potential to replace traditional anti-inflammatory drugs with drugs based on polypeptide targeting STING in the treatment of arthritis, and it has not yet been reported.

### SUMMARY OF THE INVENTION

The first objective of the present invention is to provide a polypeptide compound with anti-inflammatory activity.

The second objective of the present invention is to provide a method for preparing the polypeptide compound with anti-inflammatory activity.

The third objective of the present invention is to provide use of the polypeptide compound with anti-inflammatory activity in the preparation of a drug for the treatment of gonitis or osteoarthritis.

In order to achieve the above objectives, the present invention provides the following technical solutions.

In the first aspect, the present invention provides a polypeptide compound with anti-inflammatory activity or pharmaceutically acceptable salts thereof, wherein the polypeptide compound is screened and identified from the key residue sequences of the interaction between the two in the crystal structures of the binary complex of STING, and has a structure selected from one of the following structures:

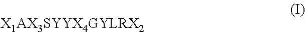

wherein
X₁ is selected from leucine or the sequence NFNVAHGL;
X₂ is selected from leucine, the sequence LIL, the sequence LILPELQ, the sequence LILPELQARIRTYN, the sequence LILPELQARIRTYNQHYNNL, the sequence LILPELQARIRTYNQHYNNLL or the sequence LILPELQARIRTYNQHYNNLLR;
X₃ is selected from tryptophan or (2R)-2-amino-2-methyl-6-heptenoic acid; and
X₄ is selected from isoleucine or (2R)-2-amino-2-methyl-6-heptenoic acid.

Preferably, paired (2R)-2-amino-2-methyl-6-heptenoic acid in the fragment is cyclized through olefin metathesis reaction.

In the polypeptide of formula (I), the amino group at the N-terminus and the carboxyl group at the C-terminus, as well as the side chain group of amino acid, may be unmodified or modified without substantially affecting the activity of the polypeptide of the present invention, for example, to form a “pharmaceutically acceptable ester”. The modification to the amino group at the N-terminus includes but is not limited to de-amination modification, N-lower-alkylation modification, N-di-lower-alkylation modification and N-acylation modification. The modification to the carboxyl group at the C-terminus includes but is not limited to amidation modification, lower-alkyl-amidation modification, dialkyl-amidation modification and lower-alkyl-esterification modification. In the polypeptide according to the present invention, the amino group at the N-terminus is subjected to acetylation modification, i.e., —Ac, and the carboxyl group at the C-terminus is subjected to amidation modification, i.e., —NH₂.

Preferably, the polypeptide compound with anti-inflammatory activity has an amino acid sequence selected from one of the following amino acid sequences:
the amino acid sequence as shown in SEQ ID NO: 1;
the amino acid sequence as shown in SEQ ID NO: 2;
the amino acid sequence as shown in SEQ ID NO: 3;
the amino acid sequence as shown in SEQ ID NO: 4;
the amino acid sequence as shown in SEQ ID NO: 5;
the amino acid sequence as shown in SEQ ID NO: 6;
the amino acid sequence as shown in SEQ ID NO: 7;
the amino acid sequence as shown in SEQ ID NO: 8;
the amino acid sequence as shown in SEQ ID NO: 9;
the amino acid sequence as shown in SEQ ID NO: 10; and
the amino acid sequence as shown in SEQ ID NO: 11.

In the second aspect, the present invention provides a method for preparing the polypeptide compound with anti-inflammatory activity, comprising the following steps:
step 1, swelling of resin: placing Fmoc-Rink amid-MBHA Resin (degree of substitution=0.44 mmol/g) in a polypeptide reaction tube, swelling with DCM, and filtrating under reduced pressure to remove DCM;
step 2, removal of Fmoc protection: adding 20% piperidine/DMF solution into the reaction tube in step 1, shaking, filtrating under reduced pressure to remove the solution, and rinsing the resin with DCM;
step 3, connection of amino acids: adding DMF to dissolve Fmoc-Leu-OH, Fmoc-Arg(Pbf)-OH, Fmoc-Asn(Trt)-OH, Fmoc-Gln(Trt)-OH or Fmoc-Gly-OH and HCTU, followed by adding DIPEA, shaking, and transferring the mixture into the polypeptide reaction tube in step 2; and shaking at room temperature, filtrating under reduced pressure to remove the solution after completion of the reaction, and rinsing the resin with DCM and DMF;
repeating step 2 and step 3 to sequentially connect Fmoc-Arg(Pbf)-OH, Fmoc-Leu-OH, Fmoc-Tyr(tBu)-OH, Fmoc-Gly-OH, Fmoc-Ile-OH, Fmoc-Tyr(tBu)-OH, Fmoc-Tyr(tBu)-OH, Fmoc-Ser(tBu)-OH, Fmoc-Trp(Boc)-OH, Fmoc-Ala-OH and Fmoc-Leu-OH, until all amino acids are connected;
or, repeating step 2 and step 3 to sequentially connect Fmoc-Arg(Pbf)-OH, Fmoc-Leu-OH, Fmoc-Tyr(tBu)-OH, Fmoc-Gly-OH, Fmoc-S₅-OH, Fmoc-Tyr(tBu)-OH, Fmoc-Tyr(tBu)-OH, Fmoc-Ser(tBu)-OH, Fmoc-S₅-OH, Fmoc-Ala-OH and Fmoc-Leu-OH, until all amino acids are connected;
or, repeating step 2 and step 3 to sequentially connect Fmoc-Leu-OH, Fmoc-Leu-OH, Fmoc-Asn(Trt)-OH, Fmoc-Asn(Trt)-OH, Fmoc-Tyr(tBu)-OH, Fmoc-His(Trt)-OH, Fmoc-Gln(Trt)-OH, Fmoc-Arg(Pbf)-OH, Fmoc-Tyr(tBu)-OH, Fmoc-Thr(tBu)-OH, Fmoc-Arg(Pbf)-OH, Fmoc-Ile-OH, Fmoc-Arg(Pbf)-OH, Fmoc-Ala-OH, Fmoc-Gln(Trt)-OH, Fmoc-Leu-OH, Fmoc-Glu(tBu)-OH, Fmoc-Pro-OH, Fmoc-Leu-OH, Fmoc-Ile-OH, Fmoc-Leu-OH, Fmoc-Arg(Pbf)-OH, Fmoc-Leu-OH and Fmoc-Tyr(tBu)-OH, until all amino acids are connected;
or, repeating step 2 and step 3 to sequentially connect Fmoc-Leu-OH, Fmoc-Leu-OH, Fmoc-Asn(Trt)-OH, Fmoc-Asn(Trt)-OH, Fmoc-Tyr(tBu)-OH, Fmoc-His(Trt)-OH, Fmoc-Gln(Trt)-OH, Fmoc-Arg(Pbf)-OH, Fmoc-Tyr(tBu)-OH, Fmoc-Thr(tBu)-OH, Fmoc-Arg(Pbf)-OH, Fmoc-Ile-OH, Fmoc-Arg(Pbf)-OH, Fmoc-Ala-OH, Fmoc-Gln(Trt)-OH, Fmoc-Leu-OH, Fmoc-Glu(tBu)-OH, Fmoc-Pro-OH, Fmoc-Leu-OH, Fmoc-Ile-OH, Fmoc-Leu-OH, Fmoc-Arg(Pbf)-OH, Fmoc-Leu-OH, Fmoc-Tyr(tBu)-OH, Fmoc-Gly-OH and Fmoc-Ile-OH, until all amino acids are connected;
or, repeating step 2 and step 3 to sequentially connect Fmoc-Ile-OH, Fmoc-Tyr(tBu)-OH, Fmoc-Tyr(tBu)-OH, Fmoc-Ser(tBu)-OH, Fmoc-Trp(Boc)-OH, Fmoc-Ala-OH, Fmoc-Leu-OH, Fmoc-Gly-OH, Fmoc-Ala-OH, Fmoc-Val-OH, Fmoc-Asn(Trt)-OH, Fmoc-Phe-OH and Fmoc-Asn(Trt)-OH, until all amino acids are connected;
or, repeating step 2 and step 3 to sequentially connect Fmoc-Ile-OH, Fmoc-Leu-OH, Fmoc-Arg(Pbf)-OH, Fmoc-Leu-OH, Fmoc-Tyr(tBu)-OH, Fmoc-Gly-OH, Fmoc-Ile-OH, Fmoc-Tyr(tBu)-OH, Fmoc-Tyr(tBu)-OH, Fmoc-Ser(tBu)-OH, Fmoc-Tyr(tBu)-OH, Fmoc-Tyr(tBu)-OH, Fmoc-Ala-OH, Fmoc-Leu-OH, Fmoc-Gly-OH, Fmoc-His(Trt)-OH, Fmoc-Ala-OH, Fmoc-Val-OH, Fmoc-Asn(Trt)-OH, Fmoc-Phe-OH and Fmoc-Asn(Trt)-OH, until all amino acids are connected;
or, repeating step 2 and step 3 to sequentially connect Fmoc-Leu-OH, Fmoc-Glu(tBu)-OH, Fmoc-Pro-OH, Fmoc-Leu-OH, Fmoc-Ile-OH, Fmoc-Leu-OH, Fmoc-Arg(Pbf)-OH, Fmoc-Leu-OH, Fmoc-Tyr(tBu)-OH, Fmoc-Gly-OH, Fmoc-Ile-OH, Fmoc-Tyr(tBu)-OH, Fmoc-Tyr(tBu)-OH, tBu)-OH, Fmoc-Ser(tBu)-OH, Fmoc-Trp(Boc)-OH, Fmoc-Ala-OH, Fmoc-Leu-OH, Fmoc-Gly-OH, Fmoc-His(Trt)-OH, Fmoc-Ala-OH, Fmoc-Val-OH, Fmoc-Asn(Trt)-OH, Fmoc-Phe-OH and Fmoc-Asn(Trt)-OH, until all amino acids are connected;
or, repeating step 2 and step 3 to sequentially connect Fmoc-Tyr(tBu)-OH, Fmoc-Thr(tBu)-OH, Fmoc-Arg(Pbf)-OH, Fmoc-Ile-OH, Fmoc-Arg(Pbf)-OH, Fmoc-Ala-OH, Fmoc-Gln(Trt)-OH, Fmoc-Leu-OH, Fmoc-Glu(tBu)-OH, Fmoc-Pro-OH, Fmoc-Leu-OH, Fmoc-Ile-OH Fmoc-Leu-OH, Fmoc-Arg(Pbf)-OH, Fmoc-Leu-OH, Fmoc-Tyr(tBu)-OH, Fmoc-Gly-OH, Fmoc-Ile-OH, Fmoc-Tyr(tBu)-OH, Fmoc-Tyr(tBu)-OH, Fmoc-Ser(tBu)-OH, Fmoc-Trp(Boc)-OH, Fmoc-Ala-OH, Fmoc-Leu-OH, Fmoc-Gly-OH, Fmoc-His(Trt)-OH, Fmoc-Ala-OH, Fmoc-Val-OH, Fmoc-Asn(Trt)-OH, Fmoc-Phe-OH and Fmoc-Asn(Trt)-OH, until all amino acids are connected;
or, repeating step 2 and step 3 to sequentially connect Fmoc-Asn(Trt)-OH, Fmoc-Asn(Trt)-OH, Fmoc-Tyr(tBu)-OH, Fmoc-His(Trt)-OH, Fmoc-Gln(Trt)-OH, Fmoc-Asn(Trt)-OH, Fmoc-Tyr(tBu)-OH, Fmoc-Thr(tBu)-OH, Fmoc-Arg(Pbf)-OH, Fmoc-Ile-OH, Fmoc-Arg(Pbf)-OH, Fmoc-Ala-OH, Fmoc-Gln(Trt)-OH, Fmoc-Leu-OH, Fmoc-Glu(tBu)-OH, Fmoc-Pro-OH, Fmoc-Leu-OH, Fmoc-Ile-OH, Fmoc-Leu-OH, Fmoc-Arg(Pbf)-OH, Fmoc-Leu-OH, Fmoc-Tyr(tBu)-OH, Fmoc-Gly-OH, Fmoc-Ile-OH, Fmoc-Tyr(tBu)-OH, Fmoc-Tyr(tBu)-OH, Fmoc-Ser(tBu)-OH, Fmoc-Trp(Boc)-OH, Fmoc-Ala-OH, Fmoc-Leu-OH, Fmoc-Gly-OH, Fmoc-His(Trt)-OH, Fmoc-Ala-OH, Fmoc-Val-OH, Fmoc-Asn(Trt)-OH, Fmoc-Phe-OH and Fmoc-Asn(Trt)-OH, until all amino acids are connected;
or, repeating step 2 and step 3 to sequentially connect Fmoc-Leu-OH, Fmoc-Asn(Trt)-OH, Fmoc-Asn(Trt)-OH, Fmoc-Tyr(tBu)-OH, Fmoc-His(Trt)-OH, Fmoc-Gln(Trt)-OH, Fmoc-Asn(Trt)-OH, Fmoc-Tyr(tBu)-OH, Fmoc-Thr(tBu)-OH, Fmoc-Arg(Pbf)-OH, Fmoc-Ile-OH, Fmoc-Arg(Pbf)-OH, Fmoc-Ala-OH, Fmoc-Gln(Trt)-OH, Fmoc-Leu-OH, Fmoc-Glu(tBu)-OH, Fmoc-Pro-OH, Fmoc-Leu-OH, Fmoc-Ile-OH, Fmoc-Leu-OH, Fmoc-Arg(Pbf)-OH, Fmoc-Leu-OH, Fmoc-Tyr(tBu)-OH, Fmoc-Gly-OH, Fmoc-Ile-OH, Fmoc-Tyr(tBu)-OH, Fmoc-Tyr(tBu)-OH, Fmoc-Ser(tBu)-OH, Fmoc-Trp(Boc)-OH, Fmoc-Ala-OH, Fmoc-Leu-OH, Fmoc-Gly-OH, Fmoc-His(Trt)-OH, Fmoc-Ala-OH, Fmoc-Val-OH, Fmoc-Asn(Trt)-OH, Fmoc-Phe-OH and Fmoc-Asn(Trt)-OH, until all amino acids are connected;
or, repeating step 2 and step 3 to sequentially connect Fmoc-Leu-OH, Fmoc-Leu-OH, Fmoc-Asn(Trt)-OH, Fmoc-Asn(Trt)-OH, Fmoc-Tyr(tBu)-OH, Fmoc-His(Trt)-OH, Fmoc-Gln(Trt)-OH, Fmoc-Asn(Trt)-OH, Fmoc-Tyr(tBu)-OH, Fmoc-Thr(tBu)-OH, Fmoc-Arg(Pbf)-OH, Fmoc-Ile-OH, Fmoc-Arg(Pbf)-OH, Fmoc-Ala-OH, Fmoc-Gln(Trt)-OH, Fmoc-Leu-OH, Fmoc-Glu(tBu)-OH, Fmoc-Pro-OH, Fmoc-Leu-OH, Fmoc-Ile-OH, Fmoc-Leu-OH, Fmoc-Arg(Pbf)-OH, Fmoc-Leu-OH, Fmoc-Tyr(tBu)-OH, Fmoc-Gly-OH, Fmoc-Ile-OH, Fmoc-Tyr(tBu)-OH, Fmoc-Tyr(tBu)-OH, Fmoc-Ser(tBu)-OH, Fmoc-Trp(Boc)-OH, Fmoc-Ala-OH, Fmoc-Leu-OH, Fmoc-Gly-OH, Fmoc-His(Trt)-OH, Fmoc-Ala-OH, Fmoc-Val-OH, Fmoc-Asn(Trt)-OH, Fmoc-Phe-OH and Fmoc-Asn(Trt)-OH, until all amino acids are connected;
step 4, acetylation of the N-terminus: adding pyridine and acetic anhydride in a volume ratio of 1:1 into the polypeptide reaction tube in step 3 above, shaking at room temperature, filtrating under reduced pressure to remove the solution after completion of the reaction, and rinsing the resin with DCM and DMF;
step 5, separation of polypeptide from resin and removal of side chain protection: adding a cleaving reagent into the polypeptide reaction tube in step 4 above, wherein the cleaving reagent is TFA/TIPs/water=95:2.5:2.5, shaking at room temperature, collecting filtrate after completion of the reaction, adding glacial ether into the filtrate to precipitate crude peptide, and filtrating to give the crude peptide; and
step 6, purification of crude peptide: dissolving the crude peptide obtained in step 5 in a mixed solution of acetonitrile and water in a volume ratio of 1:1, purifying by reversed-phase HPLC, and freeze-drying to yield the polypeptide compound with anti-inflammatory activity.

The purification conditions for the reversed-phase HPLC in step 6 are as follows: chromatographic column: Shim-pack PREP-ODS 15 UM 20×250 MM (Shimadzu, Japan); pump A: acetonitrile containing 0.1% trifluoroacetic acid; pump B: water containing 0.1% trifluoroacetic acid; flushing gradient: from 90% pump B to 0% pump B within 20 minutes; detector: dual wavelengths at 214 nm and 254 nm.

The method for preparing the polypeptide compound with anti-inflammatory activity further comprises:
olefin metathesis reaction of side chain after step 4: adding 1,2-dichloroethane solution of the first-generation Grubbs catalyst into the polypeptide reaction tube in step 4, and shaking overnight at room temperature; filtrating under reduced pressure to remove the solution after completion of the reaction, and rinsing the resin with DCM and DMF; and continuing with step 5.

In the third aspect, the present invention provides use of the polypeptide compound with anti-inflammatory activity or pharmaceutically acceptable salts thereof in the treatment of gonitis or osteoarthritis.

In the present invention, a mouse OA model was induced through medial meniscus resection. At 6 and 12 weeks after treatment, Micro-CT scanning was used to obtain the three-dimensional and coronal images of the medial tibial plateau in mice, and the OARSI score of gonitis in mice was evaluated based on HE and safranin-fast green staining. The IHC testing results show that synthesized polypeptide SIP-2 targeted can reduce the expression of MMP13 and COL2A1 positive cells in cartilaginous tissue, indicating that the polypeptide of the present invention can effectively alleviate joint cartilage damage in the mouse OA model, promote the synthesis of cartilage matrix, and inhibit osteophyte formation.

The pharmaceutically acceptable salts refer to the salt formed by some small molecule acidic or alkaline compounds and polypeptides, which can generally increase the solubility of polypeptides, and the formed salt basically does not change the activity of polypeptides. For example, generally, acids that may form salts with polypeptides of the present invention include hydrochloric acid, phosphoric acid, sulfuric acid, acetic acid, succinic acid, maleic acid and citric acid; and bases that may form salts with polypeptides of the present invention include hydroxides of alkali metals or alkali earth metals, ammonium salts and carbonates.

The anti-inflammatory effect of the polypeptide compound with anti-inflammatory activity of the present invention may be verified through conventional experimental methods, e.g., cytological experiments. In specific embodiments of the present invention, cytological experiments such as fluorescence quantitative PCR are preferred. Through this experiment, it is found that the polypeptide compound with anti-inflammatory activity of the present invention exhibits in vitro anti-inflammatory effects.

Further, the present invention provides a pharmaceutical composition with the polypeptide compound with anti-inflammatory activity as an active pharmaceutical ingredient, which can be used for anti-inflammatory therapy.

The pharmaceutical composition may comprise one or more of pharmaceutically acceptable diluents, excipients or carriers, preferably in unit dosage form, such as tablets, films, pills, capsules (including sustained or delayed release forms), powders, granules, syrups or lotion, sterile solutions for injection, suspensions or freeze-dried powder injections, aerosols or liquid sprays, automatic drop injection devices or suppositories.

The active pharmaceutical ingredient in the pharmaceutical composition may be combined with a non-toxic, pharmacologically acceptable inert carrier, such as ethanol, glycerol, water, or a combination thereof. The polypeptide compound with anti-inflammatory activity of the present invention is preferred to be used with a disinfectant aqueous solution for injection.

The pharmaceutical composition of the present invention may be administered through well-known administration routes in the art, such as oral, rectal, sublingual, pulmonary, transdermal, iontophoresis, vaginal, and nasal administration. The pharmaceutical composition of the present invention is preferred for parenteral administration, such as subcutaneous, intramuscular, or intravenous injection.

Due to the adoption of the above technical solution, the present invention has the following advantages and beneficial effects.

The polypeptide compound provided by the present invention can target STING to produce anti-inflammatory effects for the treatment of gonitis and osteoarthritis.

The present invention constructs a STING immune signaling pathway activation model by stimulating mouse RAW264.7 cells with a second messenger 2′,3′-cGAMP. The RT-qPCR analysis results show that SIP-2 reduced cGAMP-induced Ifnβ gene expression in a dose-dependent manner. Further study with western blot reveals that SIP-2 effectively reduces the activation of STING-related innate immune signaling pathways through the STING-TBK1-IRF3 signaling axis. Similarly, further validation is conducted in primary mouse chondrocytes, and the results show that SIP-2 may reduce the expression of STING in articular cartilage and delay the degeneration of articular cartilage. In a mouse DMM model, quantitative scoring of cartilage degeneration, subchondral bone change and osteophyte formation was performed using the OARSI scoring system. Combined with IHC analysis results at weeks 6 and 12, the results show that SIP-2 can significantly slow down the knee joint degeneration process in mice with OA.

### BRIEF DESCRIPTION OF DRAWINGS

FIG. 1 is a schematic diagram of the structural formula of the polypeptide compound SIP-2.

FIG. 2 is a schematic diagram of activity preliminary screening and validation data of the polypeptide compound SIP-1 of the present invention.

FIG. 3 is a schematic diagram of activity preliminary screening and validation data of the polypeptide compound SIP-2 of the present invention.

FIG. 4 is a schematic diagram of activity preliminary screening and validation data of the polypeptide compound SIP-3 of the present invention.

FIG. 5 is a schematic diagram of activity preliminary screening and validation data of the polypeptide compound SIP-4 of the present invention.

FIG. 6 is a schematic diagram of activity preliminary screening and validation data of the polypeptide compound SIP-5 of the present invention.

FIG. 7 is a schematic diagram of activity preliminary screening and validation data of the polypeptide compound SIP-6 of the present invention.

FIG. 8 is a schematic diagram of activity preliminary screening and validation data of the polypeptide compound SIP-7 of the present invention.

FIG. 9 is a schematic diagram of activity preliminary screening and validation data of the polypeptide compound SIP-8 of the present invention.

FIG. 10 is a schematic diagram of activity preliminary screening and validation data of the polypeptide compound SIP-9 of the present invention.

FIG. 11 is a schematic diagram of activity preliminary screening and validation data of the polypeptide compound SIP-10 of the present invention.

FIG. 12 is a schematic diagram of activity preliminary screening and validation data of the polypeptide compound SIP-11 of the present invention.

FIG. 13 is a schematic diagram of the cytotoxicity of SIP-2 on chondrocytes at different concentrations for 24 hours.

FIG. 14 is a schematic diagram of the cytotoxicity of SIP-2 on chondrocytes at different concentrations for 48 hours.

FIG. 15 is a schematic diagram of the protective effect of SIP-2 on a cartilage inflammatory injury model at different concentrations for 24 hours.

FIG. 16 is a schematic diagram of the protective effect of SIP-2 on a cartilage inflammatory injury model at different concentrations for 48 hours.

FIG. 17 is a schematic diagram of the regulation data of SIP-2 on the release of inflammatory factor Ifnb gene in chondrocytes.

FIG. 18 is a schematic diagram of the regulation date of SIP-2 on the release of inflammatory factor Tnf gene in chondrocytes.

FIG. 19 is a schematic diagram of the regulation date of SIP-2 on the release of inflammatory factor Il6 gene in chondrocytes.

FIG. 20 is a schematic diagram of the administration regimen of SIP-2 for the treatment of arthritis injury in mice.

FIG. 21 is a schematic diagram of the effect of SIP-2 on joint cartilage sclerosis in DMM model mice.

FIG. 22 is a schematic diagram of the quantification of the effect of SIP-2 on cartilage area at the joint in a DMM model mouse.

FIG. 23 is a schematic diagram of the quantification of the effect of SIP-2 on cartilage thickness at the joint in a DMM model mouse.

FIG. 24 is a CT scan diagram of the lower limb knee joint showing the effect of SIP-2 on the generation of osteophyte at the knee joint in the DMM model.

FIG. 25 is a schematic diagram of osteophyte grading score showing the effect of SIP-2 on the generation of osteophytes at the knee joint in the DMM model.

FIG. 26 is a schematic diagram of quantitative osteophyte grade showing the effect of SIP-2 on the generation of osteophytes at the knee joint in the DMM model.

FIG. 27 is a schematic diagram of quantitative osteophyte volume showing the effect of SIP-2 on the generation of osteophytes at the knee joint in the DMM model.

FIG. 28 is a schematic diagram of safranin-fast green staining showing the effect of SIP-2 on HE pathology and SO staining evaluation of the cartilage tissue in the DMM model.

FIG. 29 is a schematic diagram of quantitative joint area showing the effect of SIP-2 on HE pathology and SO staining evaluation of the cartilage tissue in the DMM model.

FIG. 30 is a schematic diagram of quantitative joint injury showing the effect of SIP-2 on HE pathology and SO staining evaluation of the cartilage tissue in the DMM model.

### DETAILED DESCRIPTION

In order to provide a clearer illustration of the present invention, the present invention will further be set forth in conjunction with preferred embodiments in the following. Those skilled in the art should understand that the detailed description below is explanatory rather than restrictive and should not limit the scope of protection of the present invention.

The representations for polypeptides, amino acids and chemical groups used in the present invention are well-known in the art, and please refer to the definitions listed in Table 1 for the abbreviations of amino acids. Please refer to the definition in Table 2 for the structure of special amino acid. In the present invention, amino acids generally refer to L-type amino acids, unless specified otherwise.

TABLE 1
Abbreviations of amino acids

| | Three-letter | One-letter |
|---|---|---|
| Amino Acid | abbreviation | abbreviation |
| Alanine | Ala | A |
| Arginine | Arg | R |
| Asparagine | Asn | N |
| Aspartic Acid | Asp | D |
| Cysteine Amide | Cys | C |
| Glutamine | Gln | Q |
| Glutamic Acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

TABLE 2
Abbreviation of special amino acid

| Amino Acid | Abbreviation |
|---|---|
| (2R)-2-amino-2-methyl-6-heptenoic acid | S₅ |

The names, structural formulas and mass spectrometry data of the compounds synthesized in the present invention are listed in Table 3.

TABLE 3
Names, structural formulas and mass spectrometry data of preferred polypeptide active
molecules

| Name | Structural Formula | Molecular Weight |
|---|---|---|
| SIP-1 | LAWSYYIGYLRL | [M + H]⁺ = 1558.8344 |
| | | [M + 2H]²⁺ = 779.9172 |
| | | [M + 3H]³⁺ = 520.2781 |
| SIP-2 | 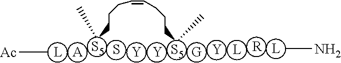 | [M + H]⁺ = 1509.8391 [M + 2H]²⁺ = 755.4195 [M + 3H]³⁺ = 503.9463 |
| SIP-3 | YLRLILPELQARIRTYNQHYNNLLR | [M + 2H]²⁺ = 1606.3945 |
| | | [M + 3H]³⁺ = 1071.1630 |
| SIP-4 | IGYLRLILPELQARIRTYNQHYNNLLR | [M + H]⁺ = 3341.8644 |
| | | [M + 2H]²⁺ = 1671.5384 |
| | | [M + 3H]³⁺ = 1114.6431 |
| SIP-5 | NFNVAHGLAWSYYIG | [M + H]⁺ = 1712.5436 |
| | | [M + 2H]²⁺ = 857.6453 |
| SIP-6 | NFNVAHGLAWSYYIGYLRLIL | [M + 2H]²⁺ = 1422.5842 |
| | | [M + 3H]³⁺ = 813.5840 |
| SIP-7 | NFNVAHGLAWSYYIGYLRLILPELQ | [M + 2H]²⁺ = 1476.2105 |
| | | [M + 3H]³⁺ = 984.6531 |
| SIP-8 | NFNVAHGLAWSYYIGYLRLILPELQARIRTYN | [M + 2H]²⁺ = 1914.2496 |
| | | [M + 3H]³⁺ = 1276.9350 |
| SIP-9 | NFNVAHGLAWSYYIGYLRLILPELQARIRTYNQHYNNL | [M + 3H]³⁺ = 1533.6832 |
| | | [M + 4H]⁴⁺ = 1150.0943 |
| SIP-10 | NFNVAHGLAWSYYIGYLRLILPELQARIRTYNQHYNNLL | [M + 3H]³⁺ = 1570.0223 |
| | | [M + 4H]⁴⁺ = 1150.8321 |
| SIP-11 | NFNVAHGLAWSYYIGYLRLILPELQARIRTYNQHYNNLLR | [M + 3H]³⁺ = 1622.1175 |
| | | [M + 4H]⁴⁺ = 1215.9946 |

### Example 1

The method for preparing straight chain polypeptide compounds with anti-inflammatory activity was provided, and the specific steps for solid-phase synthesis of SIP-1 were as follows.

### Step 1 Swelling of Resin

1 g of Fmoc-Rink amid-MBHA Resin (degree of substitution=0.44 mmol/g; GL Biochem (Shanghai) Ltd.) was placed in a polypeptide reaction tube (self-made), and swelled with 10 mL of DCM for 20 minutes. The mixture was filtrated under reduced pressure to remove DCM.

### Step 2 Removal of Fmoc Protection

The reaction tube in step 1 was added with 6 mL of 20% piperidine/DMF solution, shaking for 10 minutes. The mixture was filtrated under reduced pressure to remove the solution, and the resin was rinsed with 20 mL of DCM.

### Step 3 Connection of Amino Acids

Fmoc-Leu-OH (466 mg, 1.32 mmol) and HCTU (545 mg, 1.32 mmol) were placed in a 10 mL centrifuge tube, dissolved by adding 6 mL of DMF, and then DIPEA (170 mg, 1.32 mmol) was added. After shaking for 5 minutes, the mixture was poured into the polypeptide reaction tube in step 2, shaking at room temperature for 40 minutes. After completion of the reaction, the mixture was filtrated under reduced pressure to remove the solution, and the resin was rinsed with 20 mL of DCM and 20 mL of DMF.

Steps of removal of Fmoc protective group in step 2 and connection of amino acids in step 3 were repeated. First, 6 mL of 20% piperidine/DMF solution was added, the mixture was filtrated under reduced pressure to remove the solution after shaking for 10 minutes, and the resin was rinsed with 20 mL of DCM. Next, Fmoc-Arg(Pbf)-OH (855 mg, 1.32 mmol) and HCTU (545 mg, 1.32 mmol) were placed in a 10 mL centrifuge tube, dissolved by adding 6 mL of DMF, and then DIPEA (170 mg, 1.32 mmol) was added. After shaking for 5 minutes, the mixture was transferred into the polypeptide reaction tube in step 2, shaking at room temperature for 40 minutes. After completion of the reaction, the mixture was filtrated under reduced pressure to remove the solution, and the resin was rinsed with 20 mL of DCM and 20 mL of DMF. Fmoc-Leu-OH (466 mg, 1.32 mmol), Fmoc-Tyr(tBu)-OH (605 mg, 1.32 mmol), Fmoc-Gly-OH (392 mg, 1.32 mmol), Fmoc-Ile-OH (466 mg, 1.32 mmol), Fmoc-Tyr(tBu)-OH (605 mg, 1.32 mmol), Fmoc-Tyr(tBu)-OH (605 mg, 1.32 mmol), Fmoc-Ser(tBu)-OH (505 mg, 1.32 mmol), Fmoc-Trp(Boc)-OH (694 mg, 1.32 mmol), Fmoc-Ala-OH (410 mg, 1.32 mmol) and Fmoc-Leu-OH (466 mg, 1.32 mmol) were connected sequentially, until all amino acids were connected.

### Step 4 Acetylation of the N-Terminus

5 mL of pyridine and 5 mL of acetic anhydride were added into the polypeptide reaction tube in step 3 above, shaking at room temperature for 30 minutes. After completion of the reaction, the mixture was filtrated under reduced pressure to remove the solution, and the resin was rinsed with 20 mL of DCM and 20 mL of DMF.

Step 5 Separation of Polypeptide from Resin and Removal of Side Chain Protection

10 mL of cleaving reagent (TFA/TIPs/water=95:2.5:2.5, in volume ratio) was added into the polypeptide reaction tube in step 4 above, shaking at room temperature for 2 hours. After completion of the reaction, the filtrate was collected. 100 mL of glacial ether was added into the filtrate to precipitate crude peptide, and the mixture was filtrated to give the crude peptide.

### Step 6 Purification of Crude Peptide

0.31 g of the crude peptide obtained in step 5 was dissolved in 10 mL of a mixed solution of acetonitrile and water (1:1, in volume ratio), and purified by reversed-phase HPLC. Chromatographic column: Shim-pack PREP-ODS 15 UM 20×250 MM (Shimadzu, Japan); pump A: acetonitrile containing 0.1% trifluoroacetic acid; pump B: water containing 0.1% trifluoroacetic acid; flushing gradient: from 90% pump B to 0% pump B within 20 minutes; detector: dual wavelengths (214 nm and 254 nm). After purification, the collected liquid was freeze-dried to powder using a freeze-drying machine (Labconco, USA) to give 0.24 g of SIP-1 as white freeze-dried powder with ≥97.2% purity.

### Step 7 Identification of Polypeptide

The molecular weight of purified polypeptide was identified by high-resolution mass spectrometry (Waters Xevo G2-XS QTOF, Waters, USA). HRMS m/z [M+H]⁺=1558.8344; [M+2H]²⁺=779.9172; [M+3H]³⁺=520.2781.

The structural formula of SIP-1 is: LAWSYYIGYLRL.

### Example 2

SIP-2 was solid-phase synthesized, and the method for preparing polypeptide compound SIP-2 included the following steps.

### Step 1 Swelling of Resin

1 g of Fmoc-Rink amid-MBHA Resin (degree of substitution=0.44 mmol/g; GL Biochem (Shanghai) Ltd.) was placed in a polypeptide reaction tube (self-made), and swelled with 10 mL of DCM for 20 minutes. The mixture was filtrated under reduced pressure to remove DCM.

### Step 2 Removal of Fmoc Protection

The reaction tube in step 1 was added with 6 mL of 20% piperidine/DMF solution, shaking for 10 minutes. The mixture was filtrated under reduced pressure to remove the solution, and the resin was rinsed with 20 mL of DCM.

### Step 3 Connection of Amino Acids

Fmoc-Leu-OH (466 mg, 1.32 mmol) and HCTU (545 mg, 1.32 mmol) were placed in a 10 mL centrifuge tube, dissolved by adding 6 mL of DMF, and then DIPEA (170 mg, 1.32 mmol) was added. After shaking for 5 minutes, the mixture was poured into the polypeptide reaction tube in step 2, shaking at room temperature for 40 minutes. After completion of the reaction, the mixture was filtrated under reduced pressure to remove the solution, and the resin was rinsed with 20 mL of DCM and 20 mL of DMF.

Step 2 and step 3 were repeated to sequentially connect Fmoc-Arg(Pbf)-OH (855 mg, 1.32 mmol), Fmoc-Leu-OH (466 mg, 1.32 mmol), Fmoc-Tyr(tBu)-OH (605 mg, 1.32 mmol), Fmoc-Gly-OH (392 mg, 1.32 mmol), Fmoc-S₅—OH (350 mg, 0.88 mmol), Fmoc-Tyr(tBu)-OH (605 mg, 1.32 mmol), Fmoc-Tyr(tBu)-OH (605 mg, 1.32 mmol), Fmoc-Ser(tBu)-OH (505 mg, 1.32 mmol), Fmoc-S₅—OH (350 mg, 0.88 mmol), Fmoc-Ala-OH (410 mg, 0.88 mmol) and Fmoc-Leu-OH (466 mg, 1.32 mmol), until all amino acids were connected.

### Step 4 Acetylation of the N-Terminus

5 mL of pyridine and 5 mL of acetic anhydride were added into the polypeptide reaction tube in step 3 above, shaking at room temperature for 30 minutes. After completion of the reaction, the mixture was filtrated under reduced pressure to remove the solution, and the resin was rinsed with 20 mL of DCM and 20 mL of DMF.

### Step 5 Side Chain Olefin Metathesis Reaction

8 mL of 1,2-dichloroethane solution of the first-generation Grubbs catalyst (120 mg, 0.15 mmol) was added into the polypeptide reaction tube in step 4 above, shaking overnight at room temperature. After completion of the reaction, the mixture was filtrated under reduced pressure to remove the solution, and the resin was rinsed with 20 mL of DCM and 20 mL of DMF.

Step 6 Separation of Polypeptide from Resin and Removal of Side Chain Protection

10 mL of cleaving reagent (TFA/TIPs/water=95:2.5:2.5, in volume ratio) was added into the polypeptide reaction tube in step 5 above, shaking at room temperature for 2 hours. After completion of the reaction, the filtrate was collected. 100 mL of glacial ether was added into the filtrate to precipitate crude peptide, and the mixture was filtrated to give the crude peptide.

### Step 7 Purification of Crude Peptide

0.46 g of crude peptide obtained in step 6 was dissolved in 10 mL of a mixed solution of acetonitrile and water (1:1, in volume ratio), and purified by reversed-phase HPLC. Chromatographic column: Shim-pack PREP-ODS 15 UM 20×250 MM (Shimadzu, Japan); pump A: acetonitrile containing 0.1% trifluoroacetic acid; pump B: water containing 0.1% trifluoroacetic acid; flushing gradient: from 90% pump B to 0% pump B within 20 minutes; detector: dual wavelengths (214 nm and 254 nm). After purification, the collected liquid was freeze-dried to powder using a freeze-drying machine (Labconco, USA) to give 0.22 g of SIP-2 as white freeze-dried powder with ≥97.2% purity.

### Step 8 Identification of Polypeptide

The molecular weight of purified polypeptide was identified by high-resolution mass spectrometry (Waters Xevo G2-XS QTOF, Waters, USA). HRMS m/z: [M+H]⁺=1509.8391, [M+2H]²⁺=755.4195, [M+3H]³⁺=503.9463.

As shown in FIG. 1, FIG. 1 is a schematic diagram of the structural formula of the polypeptide compound SIP-2. The structural formula of SIP-2 is:

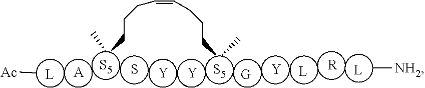

wherein S₅ is S-2-(4-pentenyl) Ala-OH. This strategy used Grubbs metathesis catalyst to immobilize amino acids containing olefins at positions i, i+4.

### Example 3

The method for preparing polypeptide compound SIP-3 included the following steps.

### Step 1 Swelling of Resin

1 g of Fmoc-Rink amid-MBHA Resin (degree of substitution=0.44 mmol/g; GL Biochem (Shanghai) Ltd.) was placed in a polypeptide reaction tube (self-made), and swelled with 10 mL of DCM for 20 minutes. The mixture was filtrated under reduced pressure to remove DCM.

### Step 2 Removal of Fmoc Protection

The reaction tube in step 1 was added with 6 mL of 20% piperidine/DMF solution, shaking for 10 minutes. The mixture was filtrated under reduced pressure to remove the solution, and the resin was rinsed with 20 mL of DCM.

### Step 3 Connection of Amino Acids

Fmoc-Arg(Pbf)-OH (855 mg, 1.32 mmol) and HCTU (545 mg, 1.32 mmol) were placed in a 10 mL centrifuge tube, dissolved by adding 6 mL of DMF, and then DIPEA (170 mg, 1.32 mmol) was added. After shaking for 5 minutes, the mixture was poured into the polypeptide reaction tube in step 2, shaking at room temperature for 40 minutes. After completion of the reaction, the mixture was filtrated under reduced pressure to remove the solution, and the resin was rinsed with 20 mL of DCM and 20 mL of DMF.

Step 2 and step 3 were repeated to sequentially connect Fmoc-Leu-OH (466 mg, 1.32 mmol), Fmoc-Leu-OH (466 mg, 1.32 mmol), Fmoc-Asn(Trt)-OH (466 mg, 1.32 mmol), Fmoc-Asn(Trt)-OH (786 mg, 1.32 mmol), Fmoc-Tyr(tBu)-OH (605 mg, 1.32 mmol), Fmoc-His(Trt)-OH (818 mg, 1.32 mmol), Fmoc-Gln(Trt)-OH (805 mg, 1.32 mmol), Fmoc-Arg(Pbf)-OH (855 mg, 1.32 mmol), Fmoc-Tyr(tBu)-OH (605 mg, 1.32 mmol), Fmoc-Thr(tBu)-OH (525 mg, 1.32 mmol), Fmoc-Arg(Pbf)-OH (855 mg, 1.32 mmol), Fmoc-Ile-OH (466 mg, 1.32 mmol), Fmoc-Arg(Pbf)-OH (855 mg, 1.32 mmol), Fmoc-Ala-OH (410 mg, 1.32 mmol), Fmoc-Gln(Trt)-OH (805 mg, 1.32 mmol), Fmoc-Leu-OH (466 mg, 1.32 mmol), Fmoc-Glu(tBu)-OH (563 mg, 1.32 mmol), Fmoc-Pro-OH (445 mg, 1.32 mmol), Fmoc-Leu-OH (466 mg, 1.32 mmol), Fmoc-Ile-OH (466 mg, 1.32 mmol), Fmoc-Leu-OH (466 mg, 1.32 mmol), Fmoc-Arg(Pbf)-OH (855 mg, 1.32 mmol), Fmoc-Leu-OH (466 mg, 1.32 mmol) and Fmoc-Tyr(tBu)-OH (605 mg, 1.32 mmol), until all amino acids were connected.

### Step 4 Acetylation of the N-Terminus

5 mL of pyridine and 5 mL of acetic anhydride were added into the polypeptide reaction tube in step 3 above, shaking at room temperature for 30 minutes. After completion of the reaction, the mixture was filtrated under reduced pressure to remove the solution, and the resin was rinsed with 20 mL of DCM and 20 mL of DMF.

Step 5 Separation of Polypeptide from Resin and Removal of Side Chain Protection

10 mL of cleaving reagent (TFA/TIPs/water=95:2.5:2.5, in volume ratio) was added into the polypeptide reaction tube in step 4 above, shaking at room temperature for 2 hours. After completion of the reaction, the filtrate was collected. 100 mL of glacial ether was added into the filtrate to precipitate crude peptide, and the mixture was filtrated to give the crude peptide.

### Step 6 Purification of Crude Peptide

0.38 g of the crude peptide obtained in step 5 was dissolved in 10 mL of a mixed solution of acetonitrile and water (1:1, in volume ratio), and purified by reversed-phase HPLC. Chromatographic column: Shim-pack PREP-ODS 15 UM 20×250 MM (Shimadzu, Japan); pump A: acetonitrile containing 0.1% trifluoroacetic acid; pump B: water containing 0.1% trifluoroacetic acid; flushing gradient: from 90% pump B to 0% pump B within 20 minutes; detector: dual wavelengths (214 nm and 254 nm). After purification, the collected liquid was freeze-dried to powder using a freeze-drying machine (Labconco, USA) to give 0.24 g of SIP-3 as white freeze-dried powder with ≥97.2% purity.

### Step 7 Identification of Polypeptide

The molecular weight of purified polypeptide was identified by high-resolution mass spectrometry (Waters Xevo G2-XS QTOF, Waters, USA). HRMS m/z: [M+2H]²⁺=1606.3945; [M+3H]³⁺=1071.1630.

The structural formula of SIP-3 is: YLRLLILPELQARIRTYNQHYNNLLR.

### Example 4

The method for preparing polypeptide compound SIP-4 included the following steps.

### Step 1 Swelling of Resin

1 g of Fmoc-Rink amid-MBHA Resin (degree of substitution=0.44 mmol/g; GL Biochem (Shanghai) Ltd.) was placed in a polypeptide reaction tube (self-made), and swelled with 10 mL of DCM for 20 minutes. The mixture was filtrated under reduced pressure to remove DCM.

### Step 2 Removal of Fmoc Protection

The reaction tube in step 1 was added with 6 mL of 20% piperidine/DMF solution, shaking for 10 minutes. The mixture was filtrated under reduced pressure to remove the solution, and the resin was rinsed with 20 mL of DCM.

### Step 3 Connection of Amino Acids

Fmoc-Arg(Pbf)-OH (855 mg, 1.32 mmol) and HCTU (545 mg, 1.32 mmol) were placed in a 10 mL centrifuge tube, dissolved by adding 6 mL of DMF, and then DIPEA (170 mg, 1.32 mmol) was added. After shaking for 5 minutes, the mixture was poured into the polypeptide reaction tube in step 2, shaking at room temperature for 40 minutes. After completion of the reaction, the mixture was filtrated under reduced pressure to remove the solution, and the resin was rinsed with 20 mL of DCM and 20 mL of DMF.

Step 2 and step 3 were repeated to sequentially connect Fmoc-Leu-OH (466 mg, 1.32 mmol), Fmoc-Leu-OH (466 mg, 1.32 mmol), Fmoc-Asn(Trt)-OH (596 mg, 1.32 mmol), Fmoc-Asn(Trt)-OH (596 mg, 1.32 mmol), Fmoc-Tyr(tBu)-OH (605 mg, 1.32 mmol), Fmoc-His(Trt)-OH (818 mg, 1.32 mmol), Fmoc-Gln(Trt)-OH (805 mg, 1.32 mmol), Fmoc-Arg(Pbf)-OH (855 mg, 1.32 mmol), Fmoc-Tyr(tBu)-OH (605 mg, 1.32 mmol), Fmoc-Thr(tBu)-OH (525 mg, 1.32 mmol), Fmoc-Arg(Pbf)-OH (855 mg, 1.32 mmol), Fmoc-Ile-OH (466 mg, 1.32 mmol), Fmoc-Arg(Pbf)-OH (855 mg, 1.32 mmol), Fmoc-Ala-OH (410 mg, 1.32 mmol), Fmoc-Gln(Trt)-OH (805 mg, 1.32 mmol), Fmoc-Leu-OH (466 mg, 1.32 mmol), Fmoc-Glu(tBu)-OH (563 mg, 1.32 mmol), Fmoc-Pro-OH (445 mg, 1.32 mmol), Fmoc-Leu-OH (466 mg, 1.32 mmol), Fmoc-Ile-OH (466 mg, 1.32 mmol), Fmoc-Leu-OH (466 mg, 1.32 mmol), Fmoc-Arg(Pbf)-OH (855 mg, 1.32 mmol), Fmoc-Leu-OH (466 mg, 1.32 mmol), Fmoc-Tyr(tBu)-OH (605 mg, 1.32 mmol), Fmoc-Gly-OH (392 mg, 1.32 mmol) and Fmoc-Ile-OH (466 mg, 1.32 mmol), until all amino acids were connected.

### Step 4 Acetylation of the N-Terminus

5 mL of pyridine and 5 mL of acetic anhydride were added into the polypeptide reaction tube in step 3 above, shaking at room temperature for 30 minutes. After completion of the reaction, the mixture was filtrated under reduced pressure to remove the solution, and the resin was rinsed with 20 mL of DCM and 20 mL of DMF.

Step 5 Separation of Polypeptide from Resin and Removal of Side Chain Protection

10 mL of cleaving reagent (TFA/TIPs/water=95:2.5:2.5, in volume ratio) was added into the polypeptide reaction tube in step 4 above, shaking at room temperature for 2 hours. After completion of the reaction, the filtrate was collected. 100 mL of glacial ether was added into the filtrate to precipitate crude peptide, and the mixture was filtrated to give the crude peptide.

### Step 6 Purification of Crude Peptide

0.68 g of the crude peptide obtained in step 5 was dissolved in 10 mL of a mixed solution of acetonitrile and water (1:1, in volume ratio), and purified by reversed-phase HPLC. Chromatographic column: Shim-pack PREP-ODS 15 UM 20×250 MM (Shimadzu, Japan); pump A: acetonitrile containing 0.1% trifluoroacetic acid; pump B: water containing 0.1% trifluoroacetic acid; flushing gradient: from 90% pump B to 0% pump B within 20 minutes; detector: dual wavelengths (214 nm and 254 nm). After purification, the collected liquid was freeze-dried to powder using a freeze-drying machine (Labconco, USA) to give 0.36 g of SIP-4 as white freeze-dried powder with ≥97.2% purity.

### Step 7 Identification of Polypeptide

The molecular weight of purified polypeptide was identified by high-resolution mass spectrometry (Waters Xevo G2-XS QTOF, Waters, USA). HRMS m/z: [M+H]⁺=3341.86444; [M+2H]²⁺=1671.5384; [M+3H]³⁺=1114.6431.

The structural formula of SIP-4: IGYLRLLILPELQARIRTYNQHYNNLLR.

### Example 5

The method for preparing polypeptide compound SIP-5 included the following steps.

### Step 1 Swelling of Resin

1 g of Fmoc-Rink amid-MBHA Resin (degree of substitution=0.44 mmol/g; GL Biochem (Shanghai) Ltd.) was placed in a polypeptide reaction tube (self-made), and swelled with 10 mL of DCM for 20 minutes. The mixture was filtrated under reduced pressure to remove DCM.

### Step 2 Removal of Fmoc Protection

The reaction tube in step 1 was added with 6 mL of 20% piperidine/DMF solution, shaking for 10 minutes. The mixture was filtrated under reduced pressure to remove the solution, and the resin was rinsed with 20 mL of DCM.

### Step 3 Connection of Amino Acids

Fmoc-Gly-OH (392 mg, 1.32 mmol) and HCTU (545 mg, 1.32 mmol) were placed in a 10 mL centrifuge tube, dissolved by adding 6 mL of DMF, and then DIPEA (170 mg, 1.32 mmol) was added. After shaking for 5 minutes, the mixture was poured into the polypeptide reaction tube in step 2, shaking at room temperature for 40 minutes. After completion of the reaction, the mixture was filtrated under reduced pressure to remove the solution, and the resin was rinsed with 20 mL of DCM and 20 mL of DMF.

Step 2 and step 3 were repeated to sequentially connect Fmoc-Ile-OH (466 mg, 1.32 mmol), Fmoc-Tyr(tBu)-OH (605 mg, 1.32 mmol), Fmoc-Tyr(tBu)-OH (605 mg, 1.32 mmol), Fmoc-Ser(tBu)-OH (505 mg, 1.32 mmol), Fmoc-Trp(Boc)-OH (694 mg, 1.32 mmol), Fmoc-Ala-OH (410 mg, 1.32 mmol), Fmoc-Leu-OH (466 mg, 1.32 mmol), Fmoc-Gly-OH (392 mg, 1.32 mmol), Fmoc-Ala-OH (410 mg, 1.32 mmol), Fmoc-Val-OH (447 mg, 1.32 mmol), Fmoc-Asn(Trt)-OH (466 mg, 1.32 mmol), Fmoc-Phe-OH (387 mg, 1.32 mmol) and Fmoc-Asn(Trt)-OH (466 mg, 1.32 mmol), until all amino acids were connected.

### Step 4 Acetylation of the N-Terminus

5 mL of pyridine and 5 mL of acetic anhydride were added into the polypeptide reaction tube in step 3 above, shaking at room temperature for 30 minutes. After completion of the reaction, the mixture was filtrated under reduced pressure to remove the solution, and the resin was rinsed with 20 mL of DCM and 20 mL of DMF.

Step 5 Separation of polypeptide from resin and removal of side chain protection 10 mL of cleaving reagent (TFA/TIPs/water=95:2.5:2.5, in volume ratio) was added into the polypeptide reaction tube in step 4 above, shaking at room temperature for 2 hours. After completion of the reaction, the filtrate was collected. 100 mL of glacial ether was added into the filtrate to precipitate crude peptide, and the mixture was filtrated to give the crude peptide.

### Step 6 Purification of Crude Peptide

0.53 g of the crude peptide obtained in step 5 was dissolved in 10 mL of a mixed solution of acetonitrile and water (1:1, in volume ratio), and purified by reversed-phase HPLC. Chromatographic column: Shim-pack PREP-ODS 15 UM 20×250 MM (Shimadzu, Japan); pump A: acetonitrile containing 0.1% trifluoroacetic acid; pump B: water containing 0.1% trifluoroacetic acid; flushing gradient: from 90% pump B to 0% pump B within 20 minutes; detector: dual wavelengths (214 nm and 254 nm). After purification, the collected liquid was freeze-dried to powder using a freeze-drying machine (Labconco, USA) to give 0.31 g of SIP-5 as white freeze-dried powder with ≥97.2% purity.

### Step 7 Identification of Polypeptide

The molecular weight of purified polypeptide was identified by high-resolution mass spectrometry (Waters Xevo G2-XS QTOF, Waters, USA). HRMS m/z: [M+H]⁺=1712.5436; [M+2H]²⁺=857.6453.

The structural formula of SIP-5 is: NFNVAHGLAWSYYIG.

### Example 6

The method for preparing polypeptide compound SIP-6 included the following steps.

### Step 1 Swelling of Resin

1 g of Fmoc-Rink amid-MBHA Resin (degree of substitution=0.44 mmol/g; GL Biochem (Shanghai) Ltd.) was placed in a polypeptide reaction tube (self-made), and swelled with 10 mL of DCM for 20 minutes. The mixture was filtrated under reduced pressure to remove DCM.

### Step 2 Removal of Fmoc Protection

The reaction tube in step 1 was added with 6 mL of 20% piperidine/DMF solution, shaking for 10 minutes. The mixture was filtrated under reduced pressure to remove the solution, and the resin was rinsed with 20 mL of DCM.

### Step 3 Connection of Amino Acids

Fmoc-Leu-OH (466 mg, 1.32 mmol) and HCTU (545 mg, 1.32 mmol) were placed in a 10 mL centrifuge tube, dissolved by adding 6 mL of DMF, and then DIPEA (170 mg, 1.32 mmol) was added. After shaking for 5 minutes, the mixture was poured into the polypeptide reaction tube in step 2, shaking at room temperature for 40 minutes. After completion of the reaction, the mixture was filtrated under reduced pressure to remove the solution, and the resin was rinsed with 20 mL of DCM and 20 mL of DMF.

Step 2 and step 3 were repeated to sequentially connect Fmoc-Ile-OH (466 mg, 1.32 mmol), Fmoc-Leu-OH (466 mg, 1.32 mmol), Fmoc-Arg(Pbf)-OH (855 mg, 1.32 mmol), Fmoc-Leu-OH (466 mg, 1.32 mmol), Fmoc-Tyr(tBu)-OH (605 mg, 1.32 mmol), Fmoc-Gly-OH (392 mg, 1.32 mmol), Fmoc-Ile-OH (466 mg, 1.32 mmol). 1.32 mmol), Fmoc-Tyr(tBu)-OH (605 mg, 1.32 mmol), Fmoc-Tyr(tBu)-OH (605 mg, 1.32 mmol), Fmoc-Ser(tBu)-OH (505 mg, 1.32 mmol), Fmoc-Tyr(tBu)-OH (694 mg, 1.32 mmol), Fmoc-Ala-OH (410 mg, 1.32 mmol), Fmoc-Leu-OH (466 mg, 1.32 mmol), Fmoc-Gly-OH (392 mg, 1.32 mmol), Fmoc-His(Trt)-OH (818 mg, 1.32 mmol), Fmoc-Ala-OH (410 mg, 1.32 mmol), Fmoc-Val-OH (447 mg, 1.32 mmol), Fmoc-Asn(Trt)-OH (786 mg, 1.32 mmol), Fmoc-Phe-OH (387 mg, 1.32 mmol) and Fmoc-Asn(Trt)-OH (786 mg, 1.32 mmol), until all amino acids were connected.

### Step 4 Acetylation of the N-Terminus

5 mL of pyridine and 5 mL of acetic anhydride were added into the polypeptide reaction tube in step 3 above, shaking at room temperature for 30 minutes. After completion of the reaction, the mixture was filtrated under reduced pressure to remove the solution, and the resin was rinsed with 20 mL of DCM and 20 mL of DMF.

Step 5 Separation of Polypeptide from Resin and Removal of Side Chain Protection

10 mL of cleaving reagent (TFA/TIPs/water=95:2.5:2.5, in volume ratio) was added into the polypeptide reaction tube in step 4 above, shaking at room temperature for 2 hours. After completion of the reaction, the filtrate was collected. 100 mL of glacial ether was added into the filtrate to precipitate crude peptide, and the mixture was filtrated to give the crude peptide.

### Step 6 Purification of Crude Peptide

0.76 g of the crude peptide obtained in step 5 was dissolved in 10 mL of a mixed solution of acetonitrile and water (1:1, in volume ratio), and purified by reversed-phase HPLC. Chromatographic column: Shim-pack PREP-ODS 15 UM 20×250 MM (Shimadzu, Japan); pump A: acetonitrile containing 0.1% trifluoroacetic acid; pump B: water containing 0.1% trifluoroacetic acid; flushing gradient: from 90% pump B to 0% pump B within 20 minutes; detector: dual wavelengths (214 nm and 254 nm). After purification, the collected liquid was freeze-dried to powder using a freeze-drying machine (Labconco, USA) to give 0.47 g of SIP-6 as white freeze-dried powder with ≥97.2% purity.

### Step 7 Identification of Polypeptide

The molecular weight of purified polypeptide was identified by high-resolution mass spectrometry (Waters Xevo G2-XS QTOF, Waters, USA). HRMS m/z: [M+2H]²⁺=1422.5842; [M+3H]³⁺=813.5840.

The structural formula of SIP-6 is: NFNVAHGLAWSYYIGYLRLIL.

### Example 7

The method for preparing polypeptide compound SIP-7 included the following steps.

### Step 1 Swelling of Resin

1 g of Fmoc-Rink amid-MBHA Resin (degree of substitution=0.44 mmol/g; GL Biochem (Shanghai) Ltd.) was placed in a polypeptide reaction tube (self-made), and swelled with 10 mL of DCM for 20 minutes. The mixture was filtrated under reduced pressure to remove DCM.

### Step 2 Removal of Fmoc Protection

The reaction tube in step 1 was added with 6 mL of 20% piperidine/DMF solution, shaking for 10 minutes. The mixture was filtrated under reduced pressure to remove the solution, and the resin was rinsed with 20 mL of DCM.

### Step 3 Connection of Amino Acids

Fmoc-Gln(Trt)-OH (805 mg, 1.32 mmol) and HCTU (545 mg, 1.32 mmol) were added into a 10 mL centrifuge tube, dissolved by adding 6 mL of DMF, and then DIPEA (170 mg, 1.32 mmol) was added. After shaking for 5 minutes, the mixture was poured into the polypeptide reaction tube in step 2, shaking at room temperature for 40 minutes. After completion of the reaction, the mixture was filtrated under reduced pressure to remove the solution, and the resin was rinsed with 20 mL of DCM and 20 mL of DMF

Step 2 and step 3 were repeated to sequentially connect Fmoc-Leu-OH (466 mg, 1.32 mmol), Fmoc-Glu(tBu)-OH (563 mg, 1.32 mmol), Fmoc-Pro-OH (445 mg, 1.32 mmol), Fmoc-Leu-OH (466 mg, 1.32 mmol), Fmoc-Ile-OH (466 mg, 1.32 mmol), Fmoc-Leu-OH (466 mg, 1.32 mmol), Fmoc-Arg(Pbf)-OH (855 mg, 1.32 mmol). 2 mmol), Fmoc-Leu-OH (466 mg, 1.32 mmol), Fmoc-Tyr(tBu)-OH (605 mg, 1.32 mmol), Fmoc-Gly-OH (392 mg, 1.32 mmol), Fmoc-Ile-OH (466 mg, 1.32 mmol), Fmoc-Tyr(tBu)-OH (605 mg, 1.32 mmol), Fmoc-Tyr(tBu)-OH (605 mg, 1.32 mmol), Fmoc-Ser(tBu)-OH (505 mg, 1.32 mmol), Fmoc-Trp(Boc)-OH (694 mg, 1.32 mmol), Fmoc-Ala-OH (410 mg, 1.32 mmol), Fmoc-Leu-OH (466 mg, 1.32 mmol), Fmoc-Gly-OH (392 mg, 1.32 mmol), Fmoc-His(Trt)-OH (818 mg, 1.32 mmol), Fmoc-Ala-OH (410 mg, 1.32 mmol), Fmoc-Val-OH (447 mg, 1.32 mmol), Fmoc-Asn(Trt)-OH (786 mg, 1.32 mmol), Fmoc-Phe-OH (387 mg, 1.32 mmol) and Fmoc-Asn(Trt)-OH (786 mg, 1.32 mmol), until all amino acids were connected.

### Step 4 Acetylation of the N-Terminus

5 mL of pyridine and 5 mL of acetic anhydride were added into the polypeptide reaction tube in step 3 above, shaking at room temperature for 30 minutes. After completion of the reaction, the mixture was filtrated under reduced pressure to remove the solution, and the resin was rinsed with 20 mL of DCM and 20 mL of DMF.

Step 5 Separation of Polypeptide from Resin and Removal of Side Chain Protection

10 mL of cleaving reagent (TFA/TIPs/water=95:2.5:2.5, in volume ratio) was added into the polypeptide reaction tube in step 4 above, shaking at room temperature for 2 hours. After completion of the reaction, the filtrate was collected. 100 mL of glacial ether was added into the filtrate to precipitate crude peptide, and the mixture was filtrated to give the crude peptide.

### Step 6 Purification of Crude Peptide

0.66 g of the crude peptide obtained in step 5 was dissolved in 10 mL of a mixed solution of acetonitrile and water (1:1, in volume ratio), and purified by reversed-phase HPLC. Chromatographic column: Shim-pack PREP-ODS 15 UM 20×250 MM (Shimadzu, Japan); pump A: acetonitrile containing 0.1% trifluoroacetic acid; pump B: water containing 0.1% trifluoroacetic acid; flushing gradient: from 90% pump B to 0% pump B within 20 minutes; detector: dual wavelengths (214 nm and 254 nm). After purification, the collected liquid was freeze-dried to powder using a freeze-drying machine (Labconco, USA) to give 0.42 g of SIP-7 as white freeze-dried powder with ≥97.2% purity.

### Step 7 Identification of Polypeptide

The molecular weight of purified polypeptide was identified by high-resolution mass spectrometry (Waters Xevo G2-XS QTOF, Waters, USA). HRMS m/z: [M+2H]²⁺=1476.2105; [M+3H]³⁺=984.6531.

The structural formula of SIP-7 is: NFNVAHGLAWSYYIGYLRLILPELQ.

### Example 8

The method for preparing polypeptide compound SIP-8 included the following steps.

### Step 1 Swelling of Resin

1 g of Fmoc-Rink amid-MBHA Resin (degree of substitution=0.44 mmol/g; GL Biochem (Shanghai) Ltd.) was placed in a polypeptide reaction tube (self-made), and swelled with 10 mL of DCM for 20 minutes. The mixture was filtrated under reduced pressure to remove DCM.

### Step 2 Removal of Fmoc Protection

The reaction tube in step 1 was added with 6 mL of 20% piperidine/DMF solution, shaking for 10 minutes. The mixture was filtrated under reduced pressure to remove the solution, and the resin was rinsed with 20 mL of DCM.

### Step 3 Connection of Amino Acids

Fmoc-Asn(Trt)-OH (800 mg, 1.32 mmol) and HCTU (545 mg, 1.32 mmol) were added into a 10 mL centrifuge tube, dissolved by adding 6 mL of DMF, and then DIPEA (170 mg, 1.32 mmol) was added. After shaking for 5 minutes, the mixture was poured into the polypeptide reaction tube in step 2, shaking at room temperature for 40 minutes. After completion of the reaction, the mixture was filtrated under reduced pressure to remove the solution, and the resin was rinsed with 20 mL of DCM and 20 mL of DMF.

Step 2 and step 3 were repeated to sequentially connect Fmoc-Tyr(tBu)-OH (605 mg, 1.32 mmol), Fmoc-Thr(tBu)-OH (525 mg, 1.32 mmol), Fmoc-Arg(Pbf)-OH (855 mg, 1.32 mmol), Fmoc-Ile-OH (466 mg, 1.32 mmol), Fmoc-Arg(Pbf)-OH (855 mg, 1.32 mmol), Fmoc-Ala-OH (410 mg, 1.32 mmol), Fmoc-Gln(Trt)-OH (805 mg, 1.32 mmol), Fmoc-Leu-OH (466 mg, 1.32 mmol), Fmoc-Glu(tBu)-OH (563 mg, 1.32 mmol), Fmoc-Pro-OH (445 mg, 1.32 mmol), Fmoc-Leu-OH (466 mg, 1.32 mmol), Fmoc-Ile-OH (466 mg, 1.32 mmol), Fmoc-Leu-OH (466 mg, 1.32 mmol), Fmoc-Arg(Pbf)-OH Fmoc-Leu-OH (855 mg, 1.32 mmol), Fmoc-Tyr(tBu)-OH (605 mg, 1.32 mmol), Fmoc-Gly-OH (392 mg, 1.32 mmol), Fmoc-Ile-OH (466 mg, 1.32 mmol), Fmoc-Tyr(tBu)-OH (605 mg, 1.32 mmol), Fmoc-Tyr(tBu)-OH (605 mg, 1.32 mmol), Fmoc-Ser(tBu)-OH (505 mg, 1.32 mmol), Fmoc-Trp(Boc)-OH (694 mg, 1.32 mmol), Fmoc-Ala-OH (410 mg, 1.32 mmol), Fmoc-Leu-OH (466 mg, 1.32 mmol), Fmoc-Gly-OH (392 mg, 1.32 mmol), Fmoc-His(Trt)-OH (818 mg, 1.32 mmol), Fmoc-Ala-OH (410 mg, 1.32 mmol), Fmoc-Val-OH (447 mg, 1.32 mmol), Fmoc-Asn(Trt)-OH (786 mg, 1.32 mmol), Fmoc-Phe-OH (387 mg, 1.32 mmol) and Fmoc-Asn(Trt)-OH (786 mg, 1.32 mmol), until all amino acids were connected.

### Step 4 Acetylation of the N-Terminus

5 mL of pyridine and 5 mL of acetic anhydride were added into the polypeptide reaction tube in step 3 above, shaking at room temperature for 30 minutes. After completion of the reaction, the mixture was filtrated under reduced pressure to remove the solution, and the resin was rinsed with 20 mL of DCM and 20 mL of DMF.

Step 5 Separation of Polypeptide from Resin and Removal of Side Chain Protection

10 mL of cleaving reagent (TFA/TIPs/water=95:2.5:2.5, in volume ratio) was added into the polypeptide reaction tube in step 4 above, shaking at room temperature for 2 hours. After completion of the reaction, the filtrate was collected. 100 mL of glacial ether was added into the filtrate to precipitate crude peptide, and the mixture was filtrated to give the crude peptide.

### Step 6 Purification of Crude Peptide

0.81 g of the crude peptide obtained in step 5 was dissolved in 10 mL of a mixed solution of acetonitrile and water (1:1, in volume ratio), and purified by reversed-phase HPLC. Chromatographic column: Shim-pack PREP-ODS 15 UM 20×250 MM (Shimadzu, Japan); pump A: acetonitrile containing 0.1% trifluoroacetic acid; pump B: water containing 0.1% trifluoroacetic acid; flushing gradient: from 90% pump B to 0% pump B within 20 minutes; detector: dual wavelengths (214 nm and 254 nm). After purification, the collected liquid was freeze-dried to powder using a freeze-drying machine (Labconco, USA) to give 0.66 g of SIP-8 as white freeze-dried powder with ≥97.2% purity.

### Step 7 Identification of Polypeptide

The molecular weight of purified polypeptide was identified by high-resolution mass spectrometry (Waters Xevo G2-XS QTOF, Waters, USA). HRMS m/z: [M+2H]²⁺=1914.2496; [M+3H]³⁺=1276.9350.

The structural formula of SIP-8 is: NFNVAHGLAWSYYIGYLRLILPELQARIRTYN.

### Example 9

The method for preparing polypeptide compound SIP-9 included the following steps.

### Step 1 Swelling of Resin

1 g of Fmoc-Rink amid-MBHA Resin (degree of substitution=0.44 mmol/g; GL Biochem (Shanghai) Ltd.) was placed in a polypeptide reaction tube (self-made), and swelled with 10 mL of DCM for 20 minutes. The mixture was filtrated under reduced pressure to remove DCM.

### Step 2 Removal of Fmoc Protection

The reaction tube in step 1 was added with 6 mL of 20% piperidine/DMF solution, shaking for 10 minutes. The mixture was filtrated under reduced pressure to remove the solution, and the resin was rinsed with 20 mL of DCM.

### Step 3 Connection of Amino Acids

Fmoc-Leu-OH (466 mg, 1.32 mmol) and HCTU (545 mg, 1.32 mmol) were added into a 10 mL centrifuge tube, dissolved by adding 6 mL of DMF, and then DIPEA (170 mg, 1.32 mmol) was added. After shaking for 5 minutes, the mixture was poured into the polypeptide reaction tube in step 2, shaking at room temperature for 40 minutes. After completion of the reaction, the mixture was filtrated under reduced pressure to remove the solution, and the resin was rinsed with 20 mL of DCM and 20 mL of DMF.

Step 2 and step 3 were repeated to sequentially connect Fmoc-Asn(Trt)-OH (786 mg, 1.32 mmol), Fmoc-Asn(Trt)-OH (786 mg, 1.32 mmol), Fmoc-Tyr(tBu)-OH (605 mg, 1.32 mmol), Fmoc-His(Trt)-OH (818 mg, 1.32 mmol), Fmoc-Gln(Trt)-OH (805 mg, 1.32 mmol), Fmoc-Asn(Trt)-OH (786 mg, 1.32 mmol), Fmoc-Asn(Trt)-OH (786 mg, 1.32 mmol). 1), Fmoc-Tyr(tBu)-OH (605 mg, 1.32 mmol), Fmoc-Thr(tBu)-OH (525 mg, 1.32 mmol), Fmoc-Arg(Pbf)-OH (855 mg, 1.32 mmol), Fmoc-Ile-OH (466 mg, 1.32 mmol), Fmoc-Arg(Pbf)-OH (855 mg, 1.32 mmol), Fmoc-Ala-OH (410 mg, 1.32 mmol), Fmoc-Gln(Trt)-OH (805 mg, 1.32 mmol), Fmoc-Leu-OH (466 mg, 1.32 mmol), Fmoc-Glu(tBu)-OH (563 mg, 1.32 mmol), Fmoc-Pro-OH (445 mg, 1.32 mmol), Fmoc-Leu-OH (466 mg, 1.32 mmol), Fmoc-Ile-OH (466 mg, 1.32 mmol), Fmoc-Leu-OH (466 mg, 1.32 mmol), Fmoc-Arg(Pbf)-OH (855 mg, 1.32 mmol) 1), Fmoc-Leu-OH (466 mg, 1.32 mmol), Fmoc-Tyr(tBu)-OH (605 mg, 1.32 mmol), Fmoc-Gly-OH (392 mg, 1.32 mmol), Fmoc-Ile-OH (466 mg, 1.32 mmol), Fmoc-Tyr(tBu)-OH (605 mg, 1.32 mmol) Fmoc-Tyr(tBu)-OH (605 mg, 1.32 mmol), Fmoc-Ser(tBu)-OH (505 mg, 1.32 mmol), Fmoc-Trp (Boc)-OH (694 mg, 1.32 mmol), Fmoc-Ala-OH (410 mg, 1.32 mmol), Fmoc-Leu-OH (466 mg, 1.32 mmol), Fmoc-Gly-OH (392 mg, 1.32 mmol), Fmoc-His(Trt)-OH (818 mg, 1.32 mmol), Fmoc-Ala-OH (410 mg, 1.32 mmol), Fmoc-Val-OH (447 mg, 1.32 mmol), Fmoc-Asn(Trt)-OH (786 mg, 1.32 mmol), Fmoc-Phe-OH (387 mg, 1.32 mmol) and Fmoc-Asn(Trt)-OH (786 mg, 1.32 mmol), until all amino acids were connected.

### Step 4 Acetylation of the N-Terminus

5 mL of pyridine and 5 mL of acetic anhydride were added into the polypeptide reaction tube in step 3 above, shaking at room temperature for 30 minutes. After completion of the reaction, the mixture was filtrated under reduced pressure to remove the solution, and the resin was rinsed with 20 mL of DCM and 20 mL of DMF.

Step 5 Separation of Polypeptide from Resin and Removal of Side Chain Protection

10 mL of cleaving reagent (TFA/TIPs/water=95:2.5:2.5, in volume ratio) was added into the polypeptide reaction tube in step 4 above, shaking at room temperature for 2 hours. After completion of the reaction, the filtrate was collected. 100 mL of glacial ether was added into the filtrate to precipitate crude peptide, and the mixture was filtrated to give the crude peptide.

### Step 6 Purification of Crude Peptide

0.86 g of the crude peptide obtained in step 5 was dissolved in 10 mL of a mixed solution of acetonitrile and water (1:1, in volume ratio), and purified by reversed-phase HPLC. Chromatographic column: Shim-pack PREP-ODS 15 UM 20×250 MM (Shimadzu, Japan); pump A: acetonitrile containing 0.1% trifluoroacetic acid; pump B: water containing 0.1% trifluoroacetic acid; flushing gradient: from 90% pump B to 0% pump B within 20 minutes; detector: dual wavelengths (214 nm and 254 nm). After purification, the collected liquid was freeze-dried to powder using a freeze-drying machine (Labconco, USA) to give 0.65 g of SIP-9 as white freeze-dried powder with ≥97.2% purity.

### Step 7 Identification of Polypeptide

The molecular weight of purified polypeptide was identified by high-resolution mass spectrometry (Waters Xevo G2-XS QTOF, Waters, USA). HRMS m/z: [M+3H]³⁺=1533.6832; [M+4H]⁴⁺=1150.0943.

The structural formula of SIP-9 is:

NFNVAHGLAWSYYIGYLRLILPELQARIRTYNQHYNNL.

### Example 10

The method for preparing polypeptide compound SIP-10 included the following steps.

### Step 1 Swelling of Resin

1 g of Fmoc-Rink amid-MBHA Resin (degree of substitution=0.44 mmol/g; GL Biochem (Shanghai) Ltd.) was placed in a polypeptide reaction tube (self-made), and swelled with 10 mL of DCM for 20 minutes. The mixture was filtrated under reduced pressure to remove DCM.

### Step 2 Removal of Fmoc Protection

The reaction tube in step 1 was added with 6 mL of 20% piperidine/DMF solution, shaking for 10 minutes. The mixture was filtrated under reduced pressure to remove the solution, and the resin was rinsed with 20 mL of DCM.

### Step 3 Connection of Amino Acids

Fmoc-Leu-OH (466 mg, 1.32 mmol) and HCTU (545 mg, 1.32 mmol) were placed in a 10 mL centrifuge tube, dissolved by adding 6 mL of DMF, and then DIPEA (170 mg, 1.32 mmol) was added. After shaking for 5 minutes, the mixture was poured into the polypeptide reaction tube in step 2, shaking at room temperature for 40 minutes. After completion of the reaction, the mixture was filtrated under reduced pressure to remove the solution, and the resin was rinsed with 20 mL of DCM and 20 mL of DMF.

Step 2 and step 3 were repeated to sequentially connect Fmoc-Leu-OH (466 mg, 1.32 mmol), Fmoc-Asn(Trt)-OH (786 mg, 1.32 mmol), Fmoc-Asn(Trt)-OH (786 mg, 1.32 mmol), Fmoc-Tyr(tBu)-OH (605 mg, 1.32 mmol), Fmoc-His(Trt)-OH (818 mg, 1.32 mmol), Fmoc-Gln(Trt)-OH (805 mg, 1.32 mmol), Fmoc-Asn(Trt)-OH (786 mg, 1.32 mmol), Fmoc-Tyr(tBu)-OH (605 mg, 1.32 mmol), Fmoc-Thr(tBu)-OH (525 mg, 1.32 mmol), Fmoc-Arg(Pbf)-OH (855 mg, 1.32 mmol), Fmoc-Ile-OH (466 mg, 1.32 mmol), Fmoc-Arg(Pbf)-OH (855 mg, 1.32 mmol), Fmoc-Ala-OH (410 mg, 1.32 mmol), Fmoc-Gln(Trt)-OH (805 mg, 1.32 mmol), Fmoc-Leu-OH (466 mg, 1.32 mmol), Fmoc-Glu(tBu)-OH (563 mg, 1.32 mmol), Fmoc-Pro-OH (445 mg, 1.32 mmol), Fmoc-Leu-OH (466 mg, 1.32 mmol), Fmoc-Ile-OH (466 mg, 1.32 mmol), Fmoc-Leu-OH (466 mg, 1.32 mmol), Fmoc-Arg(Pbf)-OH (855 mg, 1.32 mmol), Fmoc-Leu-OH (466 mg, 1.32 mmol), Fmoc-Tyr(tBu)-OH (605 mg, 1.32 mmol), Fmoc-Gly-OH (392 mg, 1.32 mmol), Fmoc-Ile-OH (466 mg, 1.32 mmol), Fmoc-Tyr(tBu)-OH (605 mg, 1.32 mmol), Fmoc-Tyr(tBu)-OH (605 mg, 1.32 mmol), Fmoc-Ser(tBu)-OH (505 mg, 1.32 mmol), Fmoc-Trp(Boc)-OH (694 mg, 1.32 mmol), Fmoc-Ala-OH (410 mg, 1.32 mmol), Fmoc-Leu-OH (466 mg, 1.32 mmol), Fmoc-Gly-OH (392 mg, 1.32 mmol), Fmoc-His(Trt)-OH (818 mg, 1.32 mmol), Fmoc-Ala-OH (410 mg, 1.32 mmol), Fmoc-Val-OH (447 mg, 1.32 mmol), Fmoc-Asn(Trt)-OH (786 mg, 1.32 mmol), Fmoc-Phe-OH (387 mg, 1.32 mmol) and Fmoc-Asn(Trt)-OH (786 mg, 1.32 mmol), until all amino acids were connected.

### Step 4 Acetylation of the N-Terminus

5 mL of pyridine and 5 mL of acetic anhydride were added into the polypeptide reaction tube in step 3 above, shaking at room temperature for 30 minutes. After completion of the reaction, the mixture was filtrated under reduced pressure to remove the solution, and the resin was rinsed with 20 mL of DCM and 20 mL of DMF.

Step 5 Separation of Polypeptide from Resin and Removal of Side Chain Protection

10 mL of cleaving reagent (TFA/TIPs/water=95:2.5:2.5, in volume ratio) was added into the polypeptide reaction tube in step 4 above, shaking at room temperature for 2 hours. After completion of the reaction, the filtrate was collected. 100 mL of glacial ether was added into the filtrate to precipitate crude peptide, and the mixture was filtrated to give the crude peptide.

### Step 6 Purification of Crude Peptide

0.78 g of the crude peptide obtained in step 5 was dissolved in 10 mL of a mixed solution of acetonitrile and water (1:1, in volume ratio), and purified by reversed-phase HPLC. Chromatographic column: Shim-pack PREP-ODS 15 UM 20×250 MM (Shimadzu, Japan); pump A: acetonitrile containing 0.1% trifluoroacetic acid; pump B: water containing 0.1% trifluoroacetic acid; flushing gradient: from 90% pump B to 0% pump B within 20 minutes; detector: dual wavelengths (214 nm and 254 nm). After purification, the collected liquid was freeze-dried to powder using a freeze-drying machine (Labconco, USA) to give 0.55 g of SIP-10 as white freeze-dried powder with ≥97.2% purity.

### Step 7 Identification of Polypeptide

The molecular weight of purified polypeptide was identified by high-resolution mass spectrometry (Waters Xevo G2-XS QTOF, Waters, USA). HRMS m/z: [M+3H]³⁺=1570.0223; [M+4H]⁴⁺=1150.8321.

The structural formula of SIP-10 is:

NFNVAHGLAWSYYIGYLRLILPELQARIRTYNQHYNNLL.

### Example 11

The method for preparing polypeptide compound SIP-11 included the following steps.

### Step 1 Swelling of Resin

1 g of Fmoc-Rink amid-MBHA Resin (degree of substitution=0.44 mmol/g; GL Biochem (Shanghai) Ltd.) was placed in a polypeptide reaction tube (self-made), and swelled with 10 mL of DCM for 20 minutes. The mixture was filtrated under reduced pressure to remove DCM.

### Step 2 Removal of Fmoc Protection

The reaction tube in step 1 was added with 6 mL of 20% piperidine/DMF solution, shaking for 10 minutes. The mixture was filtrated under reduced pressure to remove the solution, and the resin was rinsed with 20 mL of DCM.

### Step 3 Connection of Amino Acids

Fmoc-Arg(Pbf)-OH (855 mg, 1.32 mmol) and HCTU (545 mg, 1.32 mmol) were added into a 10 mL centrifuge tube, dissolved by adding 6 mL of DMF, and then DIPEA (170 mg, 1.32 mmol) was added. After shaking for 5 minutes, the mixture was poured into the polypeptide reaction tube in step 2, shaking at room temperature for 40 minutes. After completion of the reaction, the mixture was filtrated under reduced pressure to remove the solution, and the resin was rinsed with 20 mL of DCM and 20 mL of DMF.

Step 2 and step 3 were repeated to sequentially connect Fmoc-Leu-OH (466 mg, 1.32 mmol), Fmoc-Leu-OH (466 mg, 1.32 mmol), Fmoc-Asn(Trt)-OH (786 mg, 1.32 mmol), Fmoc-Asn(Trt)-OH (786 mg, 1.32 mmol), Fmoc-Tyr(tBu)-OH (605 mg, 1.32 mmol), Fmoc-His(Trt)-OH (818 mg, 1.32 mmol), Fmoc-Gln(Trt)-OH (805 mg, 1.32 mmol), Fmoc-Asn(Trt)-OH (786 mg, 1.32 mmol), Fmoc-Tyr(tBu)-OH (605 mg, 1.32 mmol), Fmoc-Thr(tBu)-OH (525 mg, 1.32 mmol), Fmoc-Arg(Pbf)-OH (855 mg, 1.32 mmol), Fmoc-Ile-OH (466 mg, 1.32 mmol), Fmoc-Arg(Pbf)-OH (855 mg, 1.32 mmol) g, 1.32 mmol), Fmoc-Ala-OH (410 mg, 1.32 mmol), Fmoc-Gln(Trt)-OH (805 mg, 1.32 mmol), Fmoc-Leu-OH (466 mg, 1.32 mmol), Fmoc-Glu(tBu)-OH (563 mg, 1.32 mmol), Fmoc-Pro-OH (445 mg, 1.32 mmol), Fmoc-Leu-OH (466 mg, 1.32 mmol), Fmoc-Ile-OH (466 mg, 1.32 mmol), Fmoc-Leu-OH (466 mg, 1.32 mmol), Fmoc-Arg(Pbf)-OH (855 mg, 1.32 mmol), Fmoc-Leu-OH (466 mg, 1.32 mmol), Fmoc-Tyr(tBu)-OH (605 mg, 1.32 mmol), Fmoc-Gly-OH (392 mg, 1.32 mmol) Fmoc-Ile-OH (466 mg, 1.32 mmol), Fmoc-Tyr(tBu)-OH (605 mg, 1.32 mmol), Fmoc-Tyr(tBu)-OH (605 mg, 1.32 mmol), Fmoc-Ser(tBu)-OH (505 mg, 1.32 mmol), Fmoc-Trp(Boc)-OH (694 mg, 1.32 mmol), Fmoc-Ala-OH (410 mg, 1.32 mmol), Fmoc-Leu-OH (466 mg, 1.32 mmol) 1) Fmoc-Gly-OH (392 mg, 1.32 mmol), Fmoc-His(Trt)-OH (818 mg, 1.32 mmol), Fmoc-Ala-OH (410 mg, 1.32 mmol), Fmoc-Val-OH (447 mg, 1.32 mmol), Fmoc-Asn(Trt)-OH (786 mg, 1.32 mmol), Fmoc-Phe-OH (387 mg, 1.32 mmol) and Fmoc-Asn(Trt)-OH (786 mg, 1.32 mmol), until all amino acids were connected.

### Step 4 Acetylation of the N-Terminus

5 mL of pyridine and 5 mL of acetic anhydride were added into the polypeptide reaction tube in step 3 above, shaking at room temperature for 30 minutes. After completion of the reaction, the mixture was filtrated under reduced pressure to remove the solution, and the resin was rinsed with 20 mL of DCM and 20 mL of DMF.

Step 5 Separation of Polypeptide from Resin and Removal of Side Chain Protection

10 mL of cleaving reagent (TFA/TIPs/water=95:2.5:2.5, in volume ratio) was added into the polypeptide reaction tube in step 4 above, shaking at room temperature for 2 hours. After completion of the reaction, the filtrate was collected. 100 mL of glacial ether was added into the filtrate to precipitate crude peptide, and the mixture was filtrated to give the crude peptide.

### Step 6 Purification of Crude Peptide

0.80 g of the crude peptide obtained in step 5 was dissolved in 10 mL of a mixed solution of acetonitrile and water (1:1, in volume ratio), and purified by reversed-phase HPLC. Chromatographic column: Shim-pack PREP-ODS 15 UM 20×250 MM (Shimadzu, Japan); pump A: acetonitrile containing 0.1% trifluoroacetic acid; pump B: water containing 0.1% trifluoroacetic acid; flushing gradient: from 90% pump B to 0% pump B within 20 minutes; detector: dual wavelengths (214 nm and 254 nm). After purification, the collected liquid was freeze-dried to powder using a freeze-drying machine (Labconco, USA) to give 0.66 g of SIP-11 as white freeze-dried powder with ≥97.2% purity.

### Step 7 Identification of Polypeptide

The molecular weight of purified polypeptide was identified by high-resolution mass spectrometry (Waters Xevo G2-XS QTOF, Waters, USA). HRMS m/z: [M+3H]³⁺=1570.0223; [M+4H]⁴⁺=1150.8321.

The structural formula of SIP-11 is:

NFNVAHGLAWSYYIGYLRLILPELQARIRTYNQHYNNLLR.

### Example 12

Real-time quantitative PCR technology was used for detecting the inhibitory activity of SIP-1 to SIP-11 on IFN-β. The specific steps were as follows.

RAW264.7 (mouse monocyte macrophage leukemia cells) was used. When the cells grew to a confluence of 80-90%, the supernatant was removed. Cells were washed twice with PBS, and digested with trypsin containing 0.25% EDTA. Upon the cells were detached, an equal volume of complete culture medium was added to terminate digestion. The digest was transferred to a 15 mL centrifuge tube to collect cells, and the supernatant was discarded. 1-2 mL of culture medium was added to resuspend cells. Cells were counted, and seeded into a 48-well plate at a density of 8000 cells per well. For the initial screening, cells were cultured in fresh medium containing polypeptide of the same concentration (10 μM) respectively for 6 hours, and then stimulated with 2′3′-cGAMP (5 μg/mL) for 4 hours. RNA was extracted with TRIzol kit, quantified, and reverse-transcribed into cDNA. Real-time quantitative PCR technology was used to detect changes in Ifnb mRNA at the mRNA level. For the efficacy validation of preferable compounds, cells were cultured in fresh culture media containing drugs at different concentrations (0, 1, 2.5, 5, 7.5, 10, 20, 40, 80 μM), and then stimulated with 2′3′-cGAMP (5 μg/mL) for 4 hours. RNA was extracted, and fluorescence quantitative PCR technology was used to detect the expression level of Ifnb mRNA. Graphpad Prism 9.0 software was used to fit the IC₅₀ value.

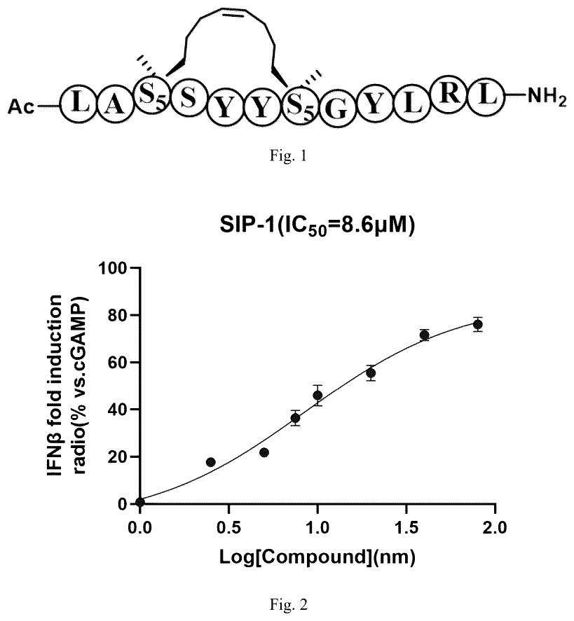

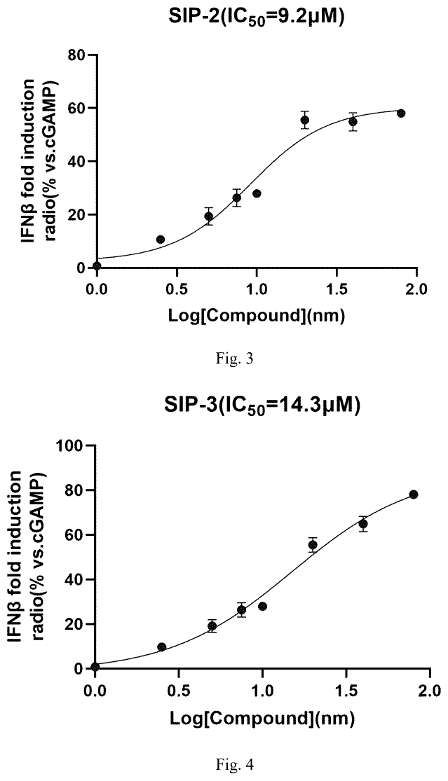

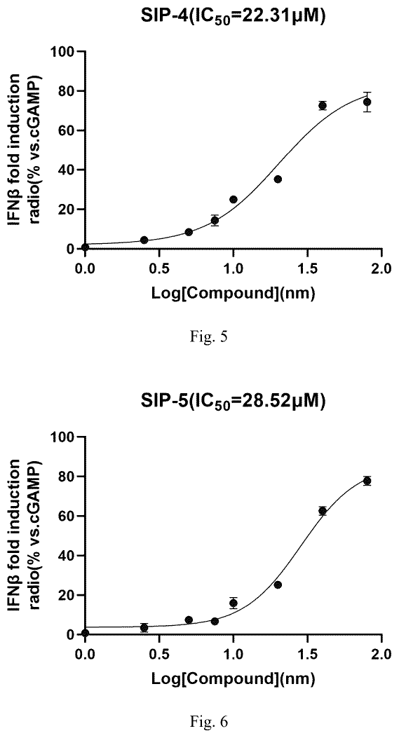

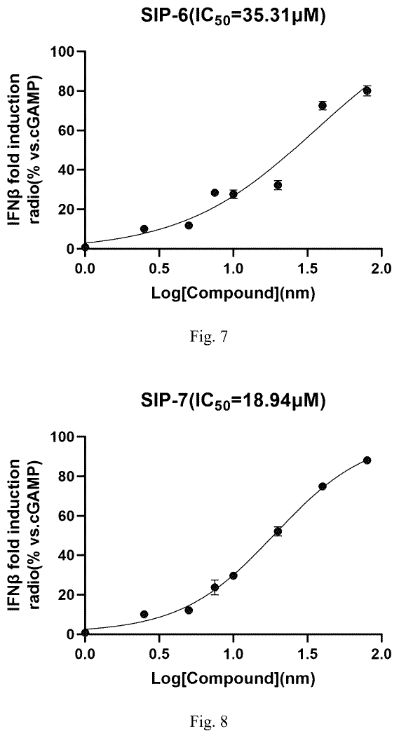

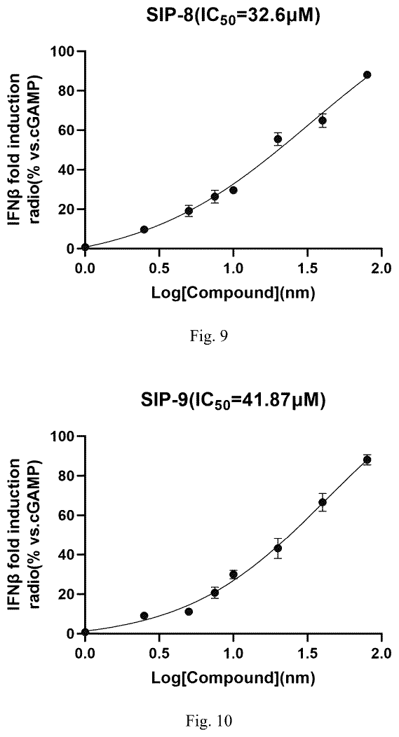

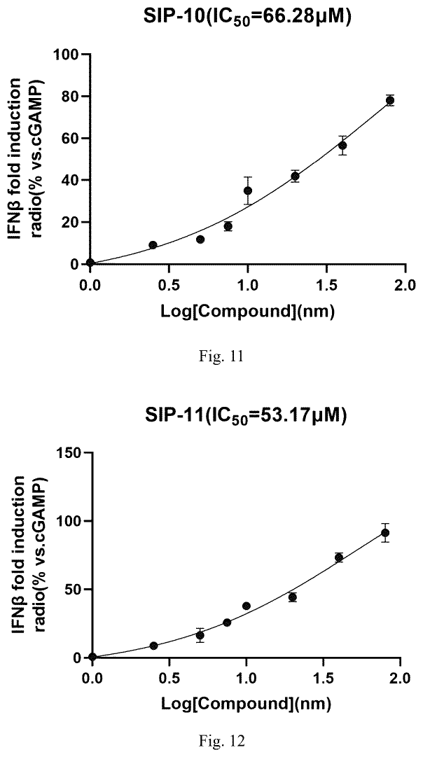

Experimental results: As shown in FIGS. 2-12, FIG. 2 was a schematic diagram of activity preliminary screening and validation data of the polypeptide compound SIP-1 of the present invention. FIG. 3 was a schematic diagram of activity preliminary screening and validation data of the polypeptide compound SIP-2 of the present invention. FIG. 4 was a schematic diagram of activity preliminary screening and validation data of the polypeptide compound SIP-3 of the present invention. FIG. 5 was a schematic diagram of activity preliminary screening and validation data of the polypeptide compound SIP-4 of the present invention. FIG. 6 was a schematic diagram of activity preliminary screening and validation data of the polypeptide compound SIP-5 of the present invention. FIG. 7 was a schematic diagram of activity preliminary screening and validation data of the polypeptide compound SIP-6 of the present invention. FIG. 8 was a schematic diagram of activity preliminary screening and validation data of the polypeptide compound SIP-7 of the present invention. FIG. 9 was a schematic diagram of activity preliminary screening and validation data of the polypeptide compound SIP-8 of the present invention. FIG. 10 was a schematic diagram of activity preliminary screening and validation data of the polypeptide compound SIP-9 of the present invention. FIG. 11 was a schematic diagram of activity preliminary screening and validation data of the polypeptide compound SIP-10 of the present invention. FIG. 12 was a schematic diagram of activity preliminary screening and validation data of the polypeptide compound SIP-11 of the present invention. The IC₅₀ values of each polypeptide compound for inhibiting Ifnb mRNA were shown in the figures: SIP-1 inhibited Ifnb mRNA with a IC₅₀ value of 8.6 μM; SIP-2 inhibited Ifnb mRNA with a IC₅₀ value of 9.2 μM; SIP-3 inhibited Ifnb mRNA with a IC₅₀ value of 14.3 μM; SIP-4 inhibited Ifnb mRNA with a IC₅₀ value of 22.31 μM; SIP-5 inhibited Ifnb mRNA with a IC₅₀ value of 28.52 μM; SIP-6 inhibited Ifnb mRNA with a IC₅₀ value of 35.31 μM; SIP-7 inhibited Ifnb mRNA with a IC₅₀ value of 18.94 μM; SIP-8 inhibited Ifnb mRNA with a IC₅₀ value of 32.6 μM; SIP-9 inhibited Ifnb mRNA with a IC₅₀ value of 41.87 μM; SIP-10 inhibited Ifnb mRNA with a IC₅₀ value of 66.28 μM; and SIP-11 inhibited Ifnb mRNA with a IC₅₀ value of 53.17 μM. All the 11 polypeptide molecules exhibited a significant inhibitory effect on the expression of Ifnb mRNA, and had a good dose-response relationship.

### Example 13

The CCK-8 assay was used to determine the toxicity of SIP-2 on chondrocytes. The specific steps were as follows.

Primary chondrocytes were extracted and passaged when the cells grew to a confluence of 80-90%. First, the culture medium was removed. Cells were washed twice with PBS, and digested with trypsin containing 0.25% EDTA. Upon the cells were detached, an equal volume of complete culture medium was added to terminate the reaction. The digest was transferred to a 15 mL centrifuge tube to collect cells, and the supernatant was discarded. 1-2 mL of culture medium was added to prepare cell suspension. The obtained chondrocytes were counted, and seeded into a 96-well plate at a density of 5000 cells per well. After overnight adhesion, cells were cultured with complete culture medium containing drugs at different concentrations (6.25, 12.5, 25, 50, 100, 200 μM) for 24 hours and 48 hours respectively. CCK-8 working solution was added, and incubation was performed in an incubator in the dark at 37° C. for 1-1.5 hours. OD values at 450 nm were detected with a microplate reader.

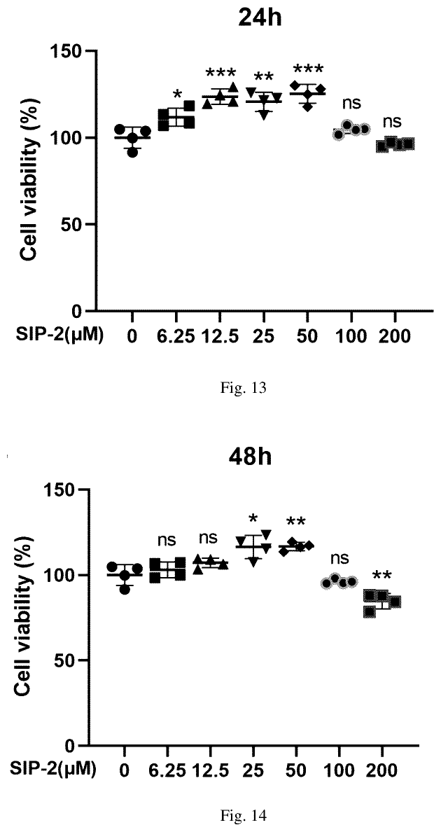

Experimental results: The results were shown in FIGS. 13 and 14. FIG. 13 was a schematic diagram of the cytotoxicity of SIP-2 on chondrocytes at different concentrations for 24 hours. FIG. 14 was a schematic diagram of the cytotoxicity of SIP-2 on chondrocytes at different concentrations for 48 hours. The figures showed that SIP-2 at a working concentration of 50 μM or below exhibited good safety for primary chondrocytes.

### Example 14

The CCK-8 assay was used to determine the protective effect of SIP-2 on chondrocyte inflammatory injury models. The specific steps were as follows.

Chondrocytes were seeded into a 96-well plate with a density of 5000 cells per well. After culturing for 24 hours, inflammatory injury models were induced with IL-1β (10 ng/ml), and co-cultured with drugs at different concentrations (10, 50 μM) for 24 and 48 hours. CCK-8 working solution was added, and incubation was performed in an incubator in the dark at 37° C. for 1-1.5 hours. OD values were detected at 450 nm with a microplate reader.

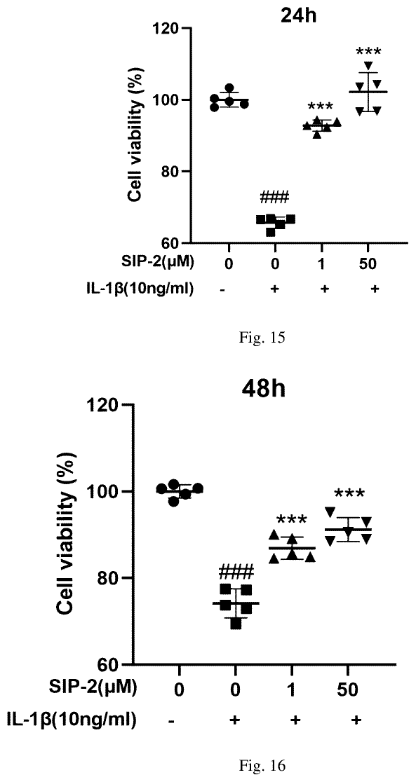

The results were shown in FIGS. 15 and 16. FIG. 15 was a schematic diagram of the protective effect of SIP-2 on a cartilage inflammatory injury model at different concentrations for 24 hours. FIG. 16 was a schematic diagram of the protective effect of SIP-2 on a cartilage inflammatory injury model at different concentrations for 48 hours. As shown in the figures, compared with the IL-1β-induced injury model, the SIP-2 drug treated group exhibited a significant increase in cell viability, indicating that SIP-2 can effectively improve cartilage inflammatory injury.

### Example 15

Real-time quantitative PCR technology was used to study the effect of SIP-2 on the mRNA changes of Ifnβ, Tnf and Il6 in chondrocytes. The specific steps were as follows.

Chondrocytes were seeded into a 48-well plate with a density of 8000 cells per well, and incubated overnight in an incubator. Cells were pre-treated by adding SIP-2 at different concentrations (10, 50 μM) for 6 hours. Inflammatory injury models were induced with IL-1β (10 ng/ml). RNA was extracted, and fluorescence quantitative PCR technology was used to detect changes in Ifnb, Tnf and Il6 genes.

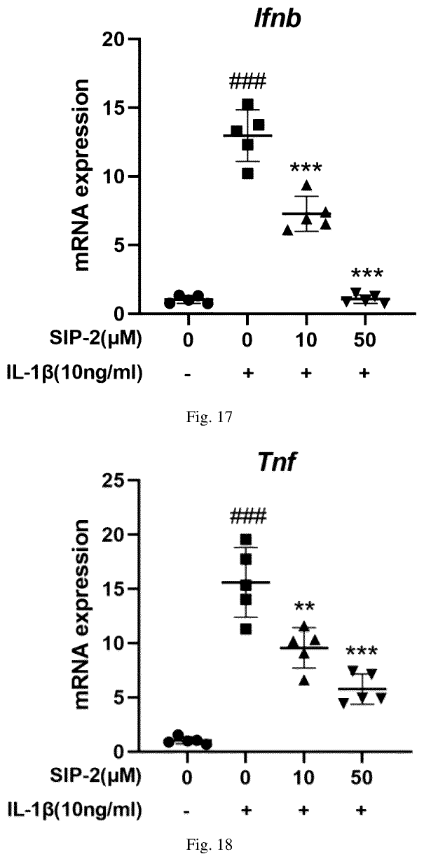

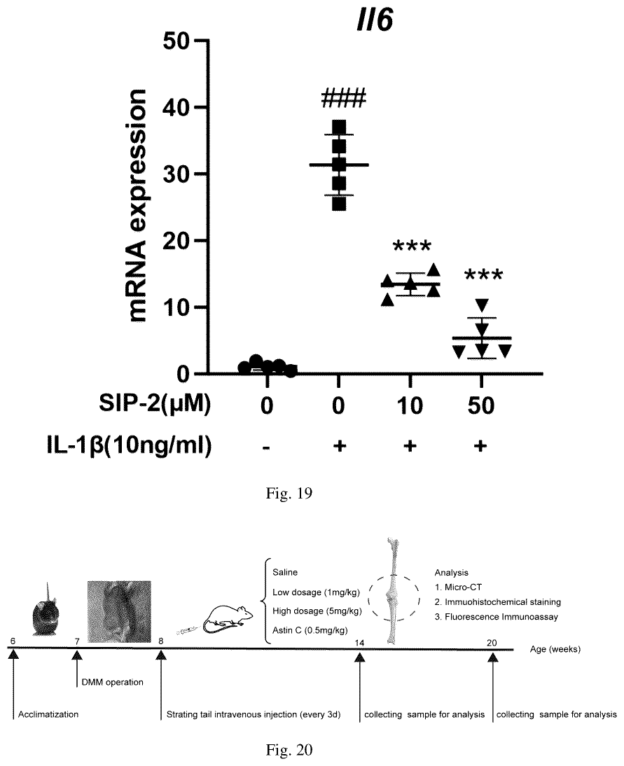

Experimental results: The results were shown in FIGS. 17-19. FIG. 17 was a schematic diagram of the regulation data of SIP-2 on the release of inflammatory factor Ifnb gene in chondrocytes. FIG. 18 was a schematic diagram of the regulation date of SIP-2 on the release of inflammatory factor Tnf gene in chondrocytes. FIG. 19 was a schematic diagram of the regulation date of SIP-2 on the release of inflammatory factor Il6 gene in chondrocytes. As shown in the figures, after stimulation with IL-1β, three factors, Ifnb, Tnf, and Il6, were significantly increased, while the transcription levels of the above inflammatory factors were significantly reduced in the SIP-2 treatment group with a good dose-response relationship. SIP-2 can improve IL-1β (10 ng/ml) induced inflammatory injury model.

### Example 16

The therapeutic effect of SIP-2 on a mouse model of arthritis induced by destabilization of medial meniscus surgery was studied. The specific steps were as follows.

Test animals: C57BL/6 mice, 36 in total, 8-9 weeks old, male, purchased from Changzhou Cavens Company.

Experimental drugs: model group (Model, n=12), low dosage group (Low dosage, 1 mg/kg, n=12), high dosage group (High dosage, 10 mg/kg, n=12).

The construction method of an arthritis model induced by destabilization of medial meniscus (DMM) surgery was as follows: mice were anesthetized with isoflurane, and fixed on the surgical table in the supine position; routine skin preparation and disinfection were performed on the left knee joint; sterilized surgical instruments were used to sequentially cut the skin and subcutaneous tissue through the medial side of the patella; the patellar ligament was gently removed, exposing the knee joint; the anterior medial meniscus ligament and medial collateral ligament were cut off, and the medial meniscus was dissociated, causing instability of the left knee joint; and the incision was sutured and disinfected with iodophor.

The experimental mice were divided into three groups: model group (Model, n=12), low dosage group (low dosage, 1 mg/kg, n=12), and high dosage group (high dosage, 10 mg/kg, n=12). The administration regimen was shown in FIG. 20 (FIG. 20 was a schematic diagram of the administration regimen of SIP-2 for the treatment of arthritis injury in mice). After DMM surgery, the mice were administered via the tail vein every three days, and joint tissue was collected for histological evaluation at 6 and 12 weeks of treatment, respectively.

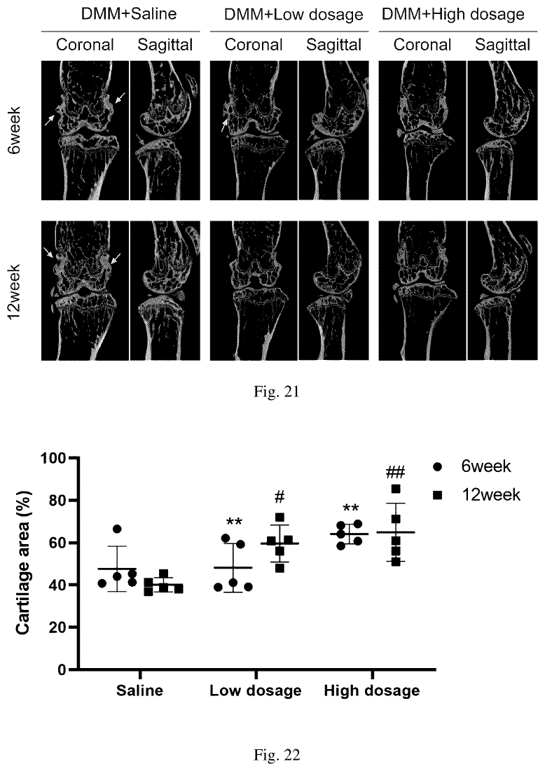

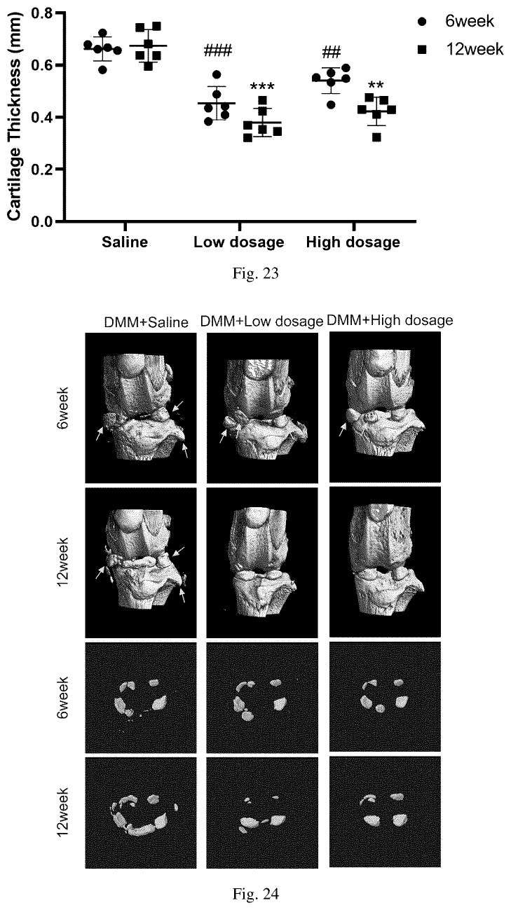

Experimental results: compared with the DMM model group, the SIP-2 treatment group (10 mg/kg) showed a widening of the knee joint space and a significant decrease in the thickness of sclerosing cartilage, as shown in FIGS. 21-23. FIG. 21 was a schematic diagram of the effect of SIP-2 on joint cartilage sclerosis in DMM model mice. FIG. 22 was a schematic diagram of the quantification of the effect of SIP-2 on cartilage area at the joint in a DMM model mouse. FIG. 23 was a schematic diagram of the quantification of the effect of SIP-2 on cartilage thickness at the joint in a DMM model mouse. As shown in the figures, SIP-2 effectively improved the process of cartilage sclerosis at the joints in DMM model mice. By observing the effect of SIP-2 on osteophytes at the joint through three-dimensional imaging, it was found that SIP-2 treatment significantly reduced the area and volume of osteophytes after DMM surgery.

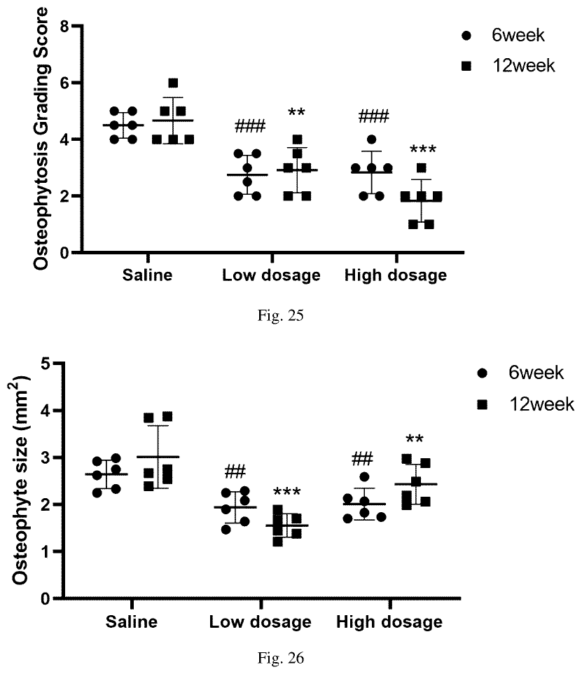

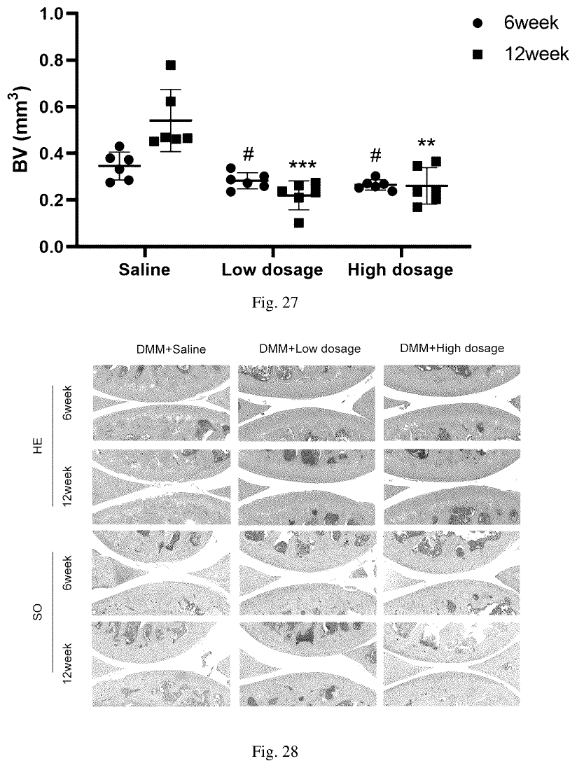

As shown in FIGS. 24-27, FIG. 24 was a CT scan diagram of the lower limb knee joint showing the effect of SIP-2 on the generation of osteophyte at the knee joint in the DMM model. FIG. 25 was a schematic diagram of osteophyte grading score showing the effect of SIP-2 on the generation of osteophytes at the knee joint in the DMM model. FIG. 26 was a schematic diagram of quantitative osteophyte grade showing the effect of SIP-2 on the generation of osteophytes at the knee joint in the DMM model. FIG. 27 was a schematic diagram of quantitative osteophyte volume showing the effect of SIP-2 on the generation of osteophytes at the knee joint in the DMM model. As shown in the figures, SIP-2 effectively improved the formation of joint osteophytes in DMM model mice. By observing the morphological changes of cartilaginous tissue, HE staining showed that compared with the model group, SIP-2 treatment resulted in smooth recovery of the cartilage surface, reduced hypertrophic chondrocytes, downward shift of tidal line, reduced distal femoral cartilage lesions and defects, intact cartilaginous tissue, and significantly increased OARSI score.

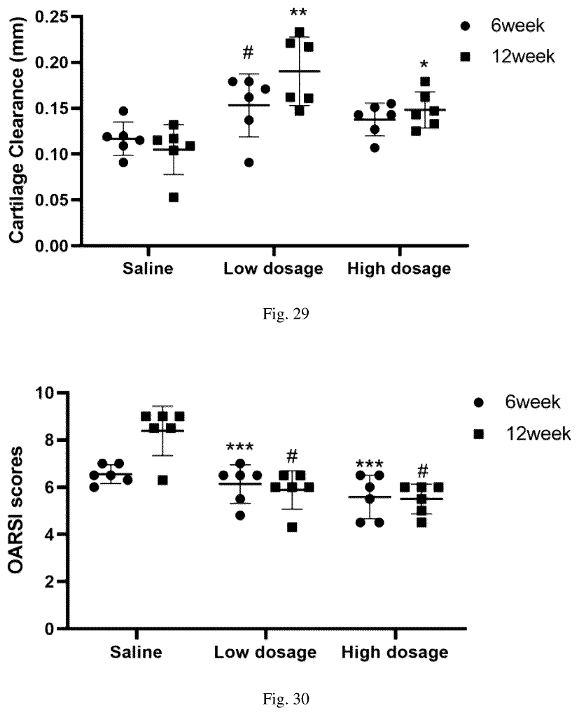

As shown in FIGS. 28-30, FIG. 28 was a schematic diagram of safranin-fast green staining showing the effect of SIP-2 on HE pathology and SO staining evaluation of the cartilage tissue in the DMM model. FIG. 29 was a schematic diagram of quantitative joint area showing the effect of SIP-2 on HE pathology and SO staining evaluation of the cartilage tissue in the DMM model. FIG. 30 was a schematic diagram of quantitative joint injury showing the effect of SIP-2 on HE pathology and SO staining evaluation of the cartilage tissue in the DMM model. As shown in the figures, the high dosage group of SIP-2 can effectively protect the normal morphology and tissue structure of cartilage at the knee joint.

The above are only preferred examples of the present invention and do not impose any formal limitations on the present invention. Although the present invention has been disclosed in preferred examples above, it is not intended to limit the present invention with the preferred examples.

What is claimed is:

1. A polypeptide compound with anti-inflammatory activity of formula (I) or pharmaceutically acceptable salts thereof: 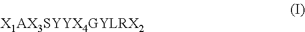
wherein
X₁ is selected from leucine or the sequence NFNVAHGL;
X₂ is selected from leucine, the sequence LIL, the sequence LILPELQ, the sequence LILPELQARIRTYN, the sequence LILPELQARIRTYNQHYNNL, the sequence LILPELQARIRTYNQHYNNLL or the sequence LILPELQARIRTYNQHYNNLLR;
X₃ is selected from tryptophan or (2R)-2-amino-2-methyl-6-heptenoic acid; and
X₄ is selected from isoleucine or (2R)-2-amino-2-methyl-6-heptenoic acid.

2. The polypeptide compound with anti-inflammatory activity or pharmaceutically acceptable salts thereof according to claim 1, wherein paired (2R)-2-amino-2-methyl-6-heptenoic acid in the polypeptide compound is cyclized through olefin metathesis reaction.

3. The polypeptide compound with anti-inflammatory activity or pharmaceutically acceptable salts thereof according to claim 1, wherein the polypeptide compound with anti-inflammatory activity has an amino acid sequence selected from one of the following amino acid sequences:
the amino acid sequence as shown in SEQ ID NO: 1;
the amino acid sequence as shown in SEQ ID NO: 2;
the amino acid sequence as shown in SEQ ID NO: 3;
the amino acid sequence as shown in SEQ ID NO: 4;
the amino acid sequence as shown in SEQ ID NO: 5;
the amino acid sequence as shown in SEQ ID NO: 6;
the amino acid sequence as shown in SEQ ID NO: 7;
the amino acid sequence as shown in SEQ ID NO: 8;
the amino acid sequence as shown in SEQ ID NO: 9;
the amino acid sequence as shown in SEQ ID NO: 10; and
the amino acid sequence as shown in SEQ ID NO: 11.

4. A method for preparing the polypeptide compound with anti-inflammatory activity according to claim 1, comprising the following steps:
step 1, swelling of resin: placing Fmoc-Rink amid-MBHA Resin in a polypeptide reaction tube, swelling with DCM, and filtrating under reduced pressure to remove DCM;
step 2, removal of Fmoc protection: adding 20% piperidine/DMF solution into the reaction tube in step 1, shaking, filtrating under reduced pressure to remove the solution, and rinsing the resin with DCM;
step 3, connection of amino acids: adding DMF to dissolve Fmoc-Leu-OH, Fmoc-Arg(Pbf)-OH, Fmoc-Asn(Trt)-OH, Fmoc-Gln(Trt)-OH or Fmoc-Gly-OH and HCTU, followed by adding DIPEA, shaking, and transferring the mixture into the polypeptide reaction tube in step 2; and shaking at room temperature, filtrating under reduced pressure to remove the solution after completion of the reaction, and rinsing the resin with DCM and DMF;
repeating step 2 and step 3 to sequentially connect Fmoc-Arg(Pbf)-OH, Fmoc-Leu-OH, Fmoc-Tyr(tBu)-OH, Fmoc-Gly-OH, Fmoc-Ile-OH, Fmoc-Tyr(tBu)-OH, Fmoc-Ser(tBu)-OH, Fmoc-Trp(Boc)-OH, Fmoc-Ala-OH and Fmoc-Leu-OH, until all amino acids are connected;
or, repeating step 2 and step 3 to sequentially connect Fmoc-Arg(Pbf)-OH, Fmoc-Leu-OH, Fmoc-Tyr(tBu)-OH, Fmoc-Gly-OH, Fmoc-S₅—OH, Fmoc-Tyr(tBu)-OH, Fmoc-Ser(tBu)-OH, Fmoc-S₅—OH, Fmoc-Ala-OH and Fmoc-Leu-OH, until all amino acids are connected;
or, repeating step 2 and step 3 to sequentially connect Fmoc-Leu-OH, Fmoc-Leu-OH, Fmoc-Asn(Trt)-OH, Fmoc-Asn(Trt)-OH, Fmoc-Tyr(tBu)-OH, Fmoc-His(Trt)-OH, Fmoc-Gln(Trt)-OH, Fmoc-Arg(Pbf)-OH, Fmoc-Tyr(tBu)-OH, Fmoc-Thr(tBu)-OH, Fmoc-Arg(Pbf)-OH, Fmoc-Ile-OH, Fmoc-Arg(Pbf)-OH, Fmoc-Ala-OH, Fmoc-Gln(Trt)-OH, Fmoc-Leu-OH, Fmoc-Glu(tBu)-OH, Fmoc-Pro-OH, Fmoc-Leu-OH, Fmoc-Ile-OH, Fmoc-Leu-OH, Fmoc-Arg(Pbf)-OH, Fmoc-Leu-OH and Fmoc-Tyr(tBu)-OH, until all amino acids are connected;
or, repeating step 2 and step 3 to sequentially connect Fmoc-Leu-OH, Fmoc-Leu-OH, Fmoc-Asn(Trt)-OH, Fmoc-Asn(Trt)-OH, Fmoc-Tyr(tBu)-OH, Fmoc-His(Trt)-OH, Fmoc-Gln(Trt)-OH, Fmoc-Arg(Pbf)-OH, Fmoc-Tyr(tBu)-OH, Fmoc-Thr(tBu)-OH, Fmoc-Arg(Pbf)-OH, Fmoc-Ile-OH, Fmoc-Arg(Pbf)-OH, Fmoc-Ala-OH, Fmoc-Gln(Trt)-OH, Fmoc-Leu-OH, Fmoc-Glu(tBu)-OH, Fmoc-Pro-OH, Fmoc-Leu-OH, Fmoc-Ile-OH, Fmoc-Leu-OH, Fmoc-Arg(Pbf)-OH, Fmoc-Leu-OH, Fmoc-Tyr(tBu)-OH, Fmoc-Gly-OH and Fmoc-Ile-OH, until all amino acids are connected;
or, repeating step 2 and step 3 to sequentially connect Fmoc-Ile-OH, Fmoc-Tyr(tBu)-OH, Fmoc-Tyr(tBu)-OH, Fmoc-Ser(tBu)-OH, Fmoc-Trp(Boc)-OH, Fmoc-Ala-OH, Fmoc-Leu-OH, Fmoc-Gly-OH, Fmoc-Ala-OH, Fmoc-Val-OH, Fmoc-Asn(Trt)-OH, Fmoc-Phe-OH and Fmoc-Asn(Trt)-OH, until all amino acids are connected;
or, repeating step 2 and step 3 to sequentially connect Fmoc-Ile-OH, Fmoc-Leu-OH, Fmoc-Arg(Pbf)-OH, Fmoc-Leu-OH, Fmoc-Tyr(tBu)-OH, Fmoc-Gly-OH, Fmoc-Ile-OH, Fmoc-Tyr(tBu)-OH, Fmoc-Tyr(tBu)-OH, Fmoc-Ser(tBu)-OH, Fmoc-Tyr(tBu)-OH, Fmoc-Tyr(tBu)-OH, Fmoc-Ala-OH, Fmoc-Leu-OH, Fmoc-Gly-OH, Fmoc-His(Trt)-OH, Fmoc-Ala-OH, Fmoc-Val-OH, Fmoc-Asn(Trt)-OH, Fmoc-Phe-OH and Fmoc-Asn(Trt)-OH, until all amino acids are connected;
or, repeating step 2 and step 3 to sequentially connect Fmoc-Leu-OH, Fmoc-Glu (tBu)-OH, Fmoc-Pro-OH, Fmoc-Leu-OH, Fmoc-Ile-OH, Fmoc-Leu-OH, Fmoc-Arg(Pbf)-OH, Fmoc-Leu-OH, Fmoc-Tyr(tBu)-OH, Fmoc-Gly-OH, Fmoc-Ile-OH, Fmoc-Tyr(tBu)-OH, Fmoc-Tyr(tBu)-OH, tBu)-OH, Fmoc-Ser(tBu)-OH, Fmoc-Trp(Boc)-OH, Fmoc-Ala-OH, Fmoc-Leu-OH, Fmoc-Gly-OH, Fmoc-His(Trt)-OH, Fmoc-Ala-OH, Fmoc-Val-OH, Fmoc-Asn(Trt)-OH, Fmoc-Phe-OH and Fmoc-Asn(Trt)-OH, until all amino acids are connected;
or, repeating step 2 and step 3 to sequentially connect Fmoc-Tyr(tBu)-OH, Fmoc-Thr(tBu)-OH, Fmoc-Arg(Pbf)-OH, Fmoc-Ile-OH, Fmoc-Arg(Pbf)-OH, Fmoc-Ala-OH, Fmoc-Gln(Trt)-OH, Fmoc-Leu-OH, Fmoc-Glu(tBu)-OH, Fmoc-Pro-OH, Fmoc-Leu-OH, Fmoc-Ile-OH Fmoc-Leu-OH, Fmoc-Arg(Pbf)-OH, Fmoc-Leu-OH, Fmoc-Tyr(tBu)-OH, Fmoc-Gly-OH, Fmoc-Ile-OH, Fmoc-Tyr(tBu)-OH, Fmoc-Tyr(tBu)-OH, Fmoc-Ser(tBu)-OH, Fmoc-Trp(Boc)-OH, Fmoc-Ala-OH, Fmoc-Leu-OH, Fmoc-Gly-OH, Fmoc-His(Trt)-OH, Fmoc-Ala-OH, Fmoc-Val-OH, Fmoc-Asn(Trt)-OH, Fmoc-Phe-OH and Fmoc-Asn(Trt)-OH, until all amino acids are connected;
or, repeating step 2 and step 3 to sequentially connect Fmoc-Asn(Trt)-OH, Fmoc-Asn(Trt)-OH, Fmoc-Tyr(tBu)-OH, Fmoc-His(Trt)-OH, Fmoc-Gln(Trt)-OH, Fmoc-Asn(Trt)-OH, Fmoc-Tyr(tBu)-OH, Fmoc-Thr(tBu)-OH, Fmoc-Arg(Pbf)-OH, Fmoc-Ile-OH, Fmoc-Arg(Pbf)-OH, Fmoc-Ala-OH, Fmoc-Gln(Trt)-OH, Fmoc-Leu-OH, Fmoc-Glu (tBu)-OH, Fmoc-Pro-OH, Fmoc-Leu-OH, Fmoc-Ile-OH, Fmoc-Leu-OH, Fmoc-Arg(Pbf)-OH, Fmoc-Leu-OH, Fmoc-Tyr(tBu)-OH, Fmoc-Gly-OH, Fmoc-Ile-OH, Fmoc-Tyr(tBu)-OH, Fmoc-Tyr(tBu)-OH, Fmoc-Ser(tBu)-OH, Fmoc-Trp(Boc)-OH, Fmoc-Ala-OH, Fmoc-Leu-OH, Fmoc-Gly-OH, Fmoc-His(Trt)-OH, Fmoc-Ala-OH, Fmoc-Val-OH, Fmoc-Asn(Trt)-OH, Fmoc-Phe-OH and Fmoc-Asn(Trt)-OH, until all amino acids are connected;
or, repeating step 2 and step 3 to sequentially connect Fmoc-Leu-OH, Fmoc-Asn(Trt)-OH, Fmoc-Asn(Trt)-OH, Fmoc-Tyr(tBu)-OH, Fmoc-His(Trt)-OH, Fmoc-Gln(Trt)-OH, Fmoc-Asn(Trt)-OH, Fmoc-Tyr(tBu)-OH, Fmoc-Thr(tBu)-OH, Fmoc-Arg(Pbf)-OH, Fmoc-Ile-OH, Fmoc-Arg(Pbf)-OH, Fmoc-Ala-OH, Fmoc-Gln(Trt)-OH, Fmoc-Leu-OH, Fmoc-Glu(tBu)-OH, Fmoc-Pro-OH, Fmoc-Leu-OH, Fmoc-Ile-OH, Fmoc-Leu-OH, Fmoc-Arg(Pbf)-OH, Fmoc-Leu-OH, Fmoc-Tyr(tBu)-OH, Fmoc-Gly-OH, Fmoc-Ile-OH, Fmoc-Tyr(tBu)-OH, Fmoc-Tyr(tBu)-OH, Fmoc-Ser(tBu)-OH, Fmoc-Trp(Boc)-OH, Fmoc-Ala-OH, Fmoc-Leu-OH, Fmoc-Gly-OH, Fmoc-His(Trt)-OH, Fmoc-Ala-OH, Fmoc-Val-OH, Fmoc-Asn(Trt)-OH, Fmoc-Phe-OH and Fmoc-Asn(Trt)-OH, until all amino acids are connected;
or, repeating step 2 and step 3 to sequentially connect Fmoc-Leu-OH, Fmoc-Leu-OH, Fmoc-Asn(Trt)-OH, Fmoc-Asn(Trt)-OH, Fmoc-Tyr(tBu)-OH, Fmoc-His(Trt)-OH, Fmoc-Gln(Trt)-OH, Fmoc-Asn(Trt)-OH, Fmoc-Tyr(tBu)-OH, Fmoc-Thr(tBu)-OH, Fmoc-Arg(Pbf)-OH, Fmoc-Ile-OH, Fmoc-Arg(Pbf)-OH, Fmoc-Ala-OH, Fmoc-Gln(Trt)-OH, Fmoc-Leu-OH, Fmoc-Glu(tBu)-OH, Fmoc-Pro-OH, Fmoc-Leu-OH, Fmoc-Ile-OH, Fmoc-Leu-OH, Fmoc-Arg(Pbf)-OH, Fmoc-Leu-OH, Fmoc-Tyr(tBu)-OH, Fmoc-Gly-OH, Fmoc-Ile-OH, Fmoc-Tyr(tBu)-OH, Fmoc-Tyr(tBu)-OH, Fmoc-Ser(tBu)-OH, Fmoc-Trp(Boc)-OH, Fmoc-Ala-OH, Fmoc-Leu-OH, Fmoc-Gly-OH, Fmoc-His(Trt)-OH, Fmoc-Ala-OH, Fmoc-Val-OH, Fmoc-Asn(Trt)-OH, Fmoc-Phe-OH and Fmoc-Asn(Trt)-OH, until all amino acids are connected;
step 4, acetylation of the N-terminus: adding pyridine and acetic anhydride in a volume ratio of 1:1 into the polypeptide reaction tube in step 3 above, shaking at room temperature, filtrating under reduced pressure to remove the solution after completion of the reaction, and rinsing the resin with DCM and DMF;
step 5, separation of polypeptide from resin and removal of side chain protection: adding a cleaving reagent into the polypeptide reaction tube in step 4 above, wherein the cleaving reagent is TFA/TIPs/water=95:2.5:2.5, shaking at room temperature, collecting filtrate after completion of the reaction, adding glacial ether into the filtrate to precipitate crude peptide, and filtrating to give the crude peptide; and
step 6, purification of crude peptide: dissolving the crude peptide obtained in step 5 in a mixed solution of acetonitrile and water in a volume ratio of 1:1, purifying by reversed-phase HPLC, and freeze-drying to yield the polypeptide compound with anti-inflammatory activity.

5. The method for preparing the polypeptide compound with anti-inflammatory activity according to claim 4, wherein the purification conditions for the reversed-phase HPLC in step 6 are as follows: chromatographic column: Shim-pack PREP-ODS 15 UM 20×250 MM; pump A: acetonitrile containing 0.1% trifluoroacetic acid; pump B: water containing 0.1% trifluoroacetic acid; flushing gradient: from 90% pump B to 0% pump B within 20 minutes; and detector: dual wavelengths at 214 nm and 254 nm.

6. The method for preparing the polypeptide compound with anti-inflammatory activity according to claim 4, wherein the method for preparing the polypeptide compound with anti-inflammatory activity further comprises:
olefin metathesis reaction of side chain after step 4: adding 1,2-dichloroethane solution of the first-generation Grubbs catalyst into the polypeptide reaction tube in step 4, and shaking overnight at room temperature; filtrating under reduced pressure to remove the solution after completion of the reaction, and rinsing the resin with DCM and DMF; and continuing with step 5.

7. Use of the polypeptide compound with anti-inflammatory activity or pharmaceutically acceptable salts thereof according to claim 1 in the treatment of gonitis or osteoarthritis.

8. Use of the polypeptide compound with anti-inflammatory activity or pharmaceutically acceptable salts thereof according to claim 2 in the treatment of gonitis or osteoarthritis.

9. A pharmaceutical composition, comprising the polypeptide compound with anti-inflammatory activity according to claim 1 as an active pharmaceutical ingredient.

10. A pharmaceutical composition, comprising the polypeptide compound with anti-inflammatory activity according to claim 2 as an active pharmaceutical ingredient.
